(19) United States
(12) Patent Application Publication
(10) Pub. No.: US 2004/0242958 A1
(43) Pub. Date: Dec. 2, 2004

(54) Endoscope apparatus

(76) Inventors: Shinji Fujikawa, Hachioji-shi (JP); Kiyoshi Miyake, Asaka-shi (JP)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/771,638

(22) Filed: Feb. 5, 2004

(30) Foreign Application Priority Data
Feb. 5, 2003 (JP) 2003-028832
Jan. 26, 2004 (JP) 2004-017452

(51) Int. Cl. A61B 1/00

(52) U.S. Cl. 600/102

(57) ABSTRACT

There is disclosed an endoscope apparatus of the present invention comprising an elongated scope section (flexible tube) partially inserted in at least an inspection object space and having flexibility and connected to an endoscope apparatus main body, and a flexible tube holding member which is detachably attached to the endoscope apparatus main body and around which the scope section is wound to hold the scope section. The flexible tube holding member is stored in a storage section detachably attached to the endoscope apparatus main body.

### CROSS-REFERENCE TO RELATED APPLICATIONS

This application is based upon and claims the benefit of priority from prior Japanese Patent Applications No. 2003-028832, filed Feb. 5, 2003; and No. 2004-017452, filed Jan. 26, 2004, the entire contents of both of which are incorporated herein by reference.

### BACKGROUND OF THE INVENTION

The present invention relates to an endoscope apparatus for use mainly in an industrial field for observing the inside of an inspection object space.

In general, an industrial endoscope apparatus has been known including a longitudinal insertion portion, with which an endoscopic inspection is performed in order to observe deep portions of industrial machines such as a plant. Since the longitudinal insertion portion is attached to the endoscope apparatus, the apparatus has not been conveniently carried or stored as such.

To solve the problem, for example, a drum type endoscope apparatus has been developed including a drum around which the longitudinal insertion portion is wound in an endoscope storage case for storing an endoscope apparatus main body. The longitudinal insertion portion is wound around the drum so that the apparatus is usable with a required insertion portion length.

As this type of endoscope apparatus, for example, in Jpn. Pat. Appln. KOKAI Publication No. 2001-330783, the drum type endoscope apparatus has been described including a constitution in which the longitudinal insertion portion is wound around the drum and the drum is disposed in the endoscope storage case for containing the endoscope main body and the insertion portion is usable with the required insertion portion length. In this constitution, the endoscope apparatus main body is drawn out of the drum at a use time of the endoscope, and is wound around the drum at a storage time.

### BRIEF SUMMARY OF THE INVENTION

According to the present invention, there is provided an endoscope apparatus including: an endoscope including an elongated flexible tube whose part is inserted in at least an inspection object space and which has flexibility; an endoscope apparatus main body connected to the endoscope for use; and a flexible tube holding member which is detachably attached to the endoscope apparatus main body and around which the flexible tube is wound to hold the flexible tube.

Furthermore, there is provided an endoscope apparatus including: an endoscope apparatus main body connected to the endoscope including an elongated flexible tube whose part is inserted in at least an inspection object space; a flexible tube holding member around which the flexible tube is wound to hold the flexible tube; and a storage section which stores the flexible tube holding member and which is detachably attached to the endoscope apparatus main body.

### BRIEF DESCRIPTION OF THE SEVERAL VIEWS OF THE DRAWING

FIG. 1A is a perspective view schematically showing a constitution of a whole endoscope apparatus main body in a state in which a lid of an endoscope storage case is opened in an endoscope apparatus according to a first embodiment of the present invention, FIG. 1B is a plan view showing a shoulder belt storage state of an industrial endoscope, and FIG. 1C is a perspective view showing a spare lamp storage section of the endoscope storage case;

FIG. 2A is a perspective view showing the endoscope storage case of the endoscope apparatus of the first embodiment, FIG. 2B is a perspective view showing the endoscope apparatus main body, FIG. 2C is a perspective view showing a storage section attachment member of the endoscope apparatus main body, and FIG. 2D is a diagram showing a constitution in which a storage section is fixed to the apparatus main body;

FIG. 3A is a plan view of a storage section in the endoscope apparatus of the first embodiment, and FIG. 3B is a sectional view along a line IIIB-IIIB of FIG. 3A;

FIG. 4A is a side view of a holding member in the endoscope apparatus of the first embodiment, and FIG. 4B is a front view of the holding member;

FIG. 5A is a plan view of a main part showing a state in which the storage section is assembled onto the endoscope apparatus main body of the first embodiment, FIG. 5B is a vertical sectional view of the storage section showing a pull-out start state of an insertion portion, and FIG. 5C is a vertical sectional view of a storage section showing a state in which an insertion portion pull-out operation proceeds;

FIG. 6 is a perspective view showing a state in which a tip portion of the insertion portion is inserted in a tip holder of the holding member in the endoscope apparatus of the first embodiment;

FIG. 7A is a vertical sectional view of the holding member in the endoscope apparatus of the first embodiment, and FIG. 7B is a sectional view along line VIIB-VIIB of FIG. 7A;

FIG. 8 is a vertical sectional view of a main part showing the endoscope apparatus of a second embodiment of the present invention;

FIG. 9A is a side view of the holding member in the endoscope apparatus of a third embodiment of the present invention, FIG. 9B is a front view of the holding member, and FIG. 9C is a perspective view of the holding member which is obliquely seen;

FIG. 10A is a plan view of the main part showing a state in which the storage section is assembled onto the endoscope apparatus main body of a fourth embodiment of the present invention, FIG. 10B is a vertical sectional view of the storage section showing a state in which the storage section is disposed on the holding member, and FIG. 10C is a vertical sectional view of the storage section showing an insertion portion pull-out operation state;

FIG. 11A is a perspective view showing the endoscope storage case in the endoscope apparatus of a fifth embodiment of the present invention, FIG. 11B is a perspective view showing the endoscope apparatus main body;

FIG. 12 is a perspective view showing the endoscope apparatus main body of the endoscope apparatus of the fifth embodiment;

FIG. 13 is a front view showing the holding member of the endoscope apparatus of the fifth embodiment;

FIG. 14 is a perspective view showing the holding member of the endoscope apparatus of a sixth embodiment of the present invention;

FIG. 15A is a perspective view showing the holding member in a seventh embodiment of the present invention, and FIG. 15B is a vertical sectional view of the holding member;

FIG. 16A is a perspective view showing the holding member in an eighth embodiment of the present invention, and FIG. 16B is a vertical sectional view of the holding member;

FIG. 17A is a perspective view showing the holding member in a ninth embodiment of the present invention, and FIG. 17B is a vertical sectional view showing the holding member of a tenth embodiment of the present invention;

FIG. 18 is a perspective view showing a state in which the holding member of the endoscope apparatus of an eleventh embodiment of the present invention is stored in the storage section;

FIG. 19A is a perspective view showing a state in which the holding member of the endoscope apparatus of a twelfth embodiment of the present invention is stored in the storage section, and FIG. 19B is a perspective view showing a modification of the storage section of FIG. 19A;

FIG. 20A is a perspective view showing a state in which the holding member of the endoscope apparatus of a thirteenth embodiment of the present invention is stored in the storage section, and FIG. 20B is a perspective view showing a modification of the storage section of FIG. 20A;

FIG. 21A is a perspective view showing the storage section of the endoscope apparatus in a fourteenth embodiment of the present invention, and FIG. 21B is a perspective view showing the holding member;

FIG. 22A is a perspective view showing the holding member of the endoscope apparatus of a fifteenth embodiment of the present invention; FIG. 22B is a perspective view showing the holding member of the endoscope apparatus of a sixteenth embodiment of the present invention, and FIG. 22C is a perspective view showing the holding member of the endoscope apparatus of a seventeenth embodiment of the present invention;

FIG. 23 is a perspective view showing the endoscope apparatus main body of the endoscope apparatus of an eighteenth embodiment of the present invention;

FIG. 24 is a perspective view showing a state in which the holding member is detached from the endoscope apparatus main body of the endoscope apparatus of the eighteenth embodiment;

FIG. 25 is a perspective view showing the endoscope apparatus main body of the endoscope apparatus of a nineteenth embodiment of the present invention;

FIG. 26 is a perspective view showing a state in which the holding member is detached from the endoscope apparatus main body of the endoscope apparatus of the nineteenth embodiment;

FIG. 27 is a perspective view showing the holding member of the endoscope apparatus of a twentieth embodiment of the present invention;

FIG. 28A is a perspective view showing the holding member of the endoscope apparatus of a twenty-first embodiment of the present invention, and FIG. 28B is a perspective view showing a modification of the holding member of the twenty-first embodiment;

FIG. 29 is a perspective view showing the holding member of the endoscope apparatus of a twenty-second embodiment of the present invention;

FIGS. 30A, 30B, 30C are diagrams showing an appearance constitution of the endoscope storage case of the endoscope apparatus of a twenty-third embodiment of the present invention;

FIGS. 31A, 31B, 31C are perspective views showing the holding member in a twenty-fourth embodiment of the present invention, and FIG. 31D is a diagram showing a constitution example of the endoscope apparatus main body;

FIG. 32A is a perspective view showing a state in which the holding member is removed from the endoscope apparatus main body in the twenty-fourth embodiment, FIG. 32B is a perspective view showing the holding member, and FIG. 32C is a perspective view showing a state in which the holding member is stored;

FIG. 33A is an explanatory view showing that a scope section is fitted into the holding member in the twenty-fifth embodiment of the present invention, and FIG. 33B is a perspective view showing the endoscope apparatus main body, holding member, and endoscope storage case;

FIG. 34 is a perspective view showing the endoscope storage case in which the endoscope apparatus main body and holding member are stored in the twenty-fifth embodiment;

FIG. 35A is a perspective view showing the holding member in a twenty-sixth embodiment of the present invention, and FIG. 35B is a perspective view showing the endoscope apparatus main body, holding member, and endoscope storage case;

FIG. 36 is a perspective view showing the endoscope apparatus including a long universal cable in a twenty-seventh embodiment of the present invention;

FIG. 37A is a perspective view showing the holding member with which the universal cable is wound and the endoscope apparatus main body in a twenty-eighth embodiment, and FIG. 37B is a perspective view showing the storage section in which the holding member is stored and the endoscope apparatus main body;

FIG. 38 is a perspective view showing the endoscope apparatus main body of the endoscope apparatus of a twenty-ninth embodiment of the present invention;

FIG. 39 is a diagram showing a first modification in the twenty-ninth embodiment;

FIG. 40A is a diagram showing a second modification in the twenty-ninth embodiment, FIG. 40B is a diagram showing a third modification, and FIG. 40C is a diagram showing a fourth modification; and

FIG. 41 is a diagram showing a fifth modification in the twenty-ninth embodiment.

### DETAILED DESCRIPTION OF THE INVENTION

Embodiments of the present invention will be described hereinafter in detail with reference to the drawings.

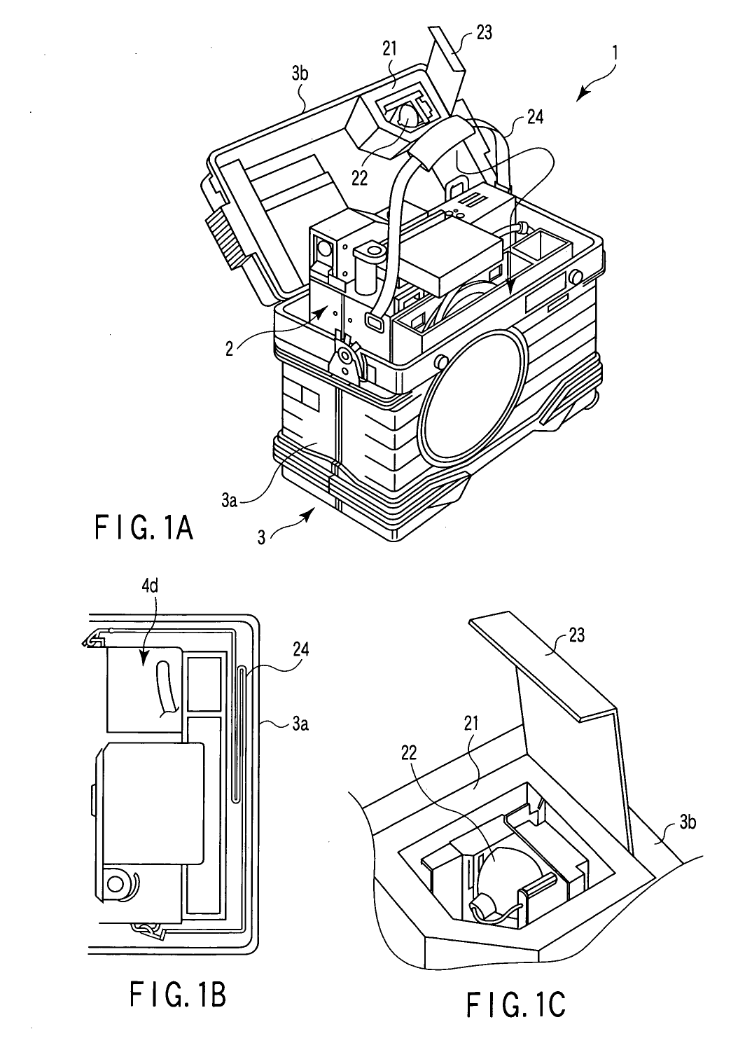

A first embodiment of the present invention will be described with reference to FIGS. 1A to 1C to 7A, 7B. FIG. 1A shows a schematic constitution of a whole endoscope apparatus 1 of the present embodiment. In this endoscope apparatus 1, an apparatus main body 2 onto which constituting elements of an endoscope are integrally assembled, and an endoscope storage case 3 in which the apparatus main body 2 is detachably stored.

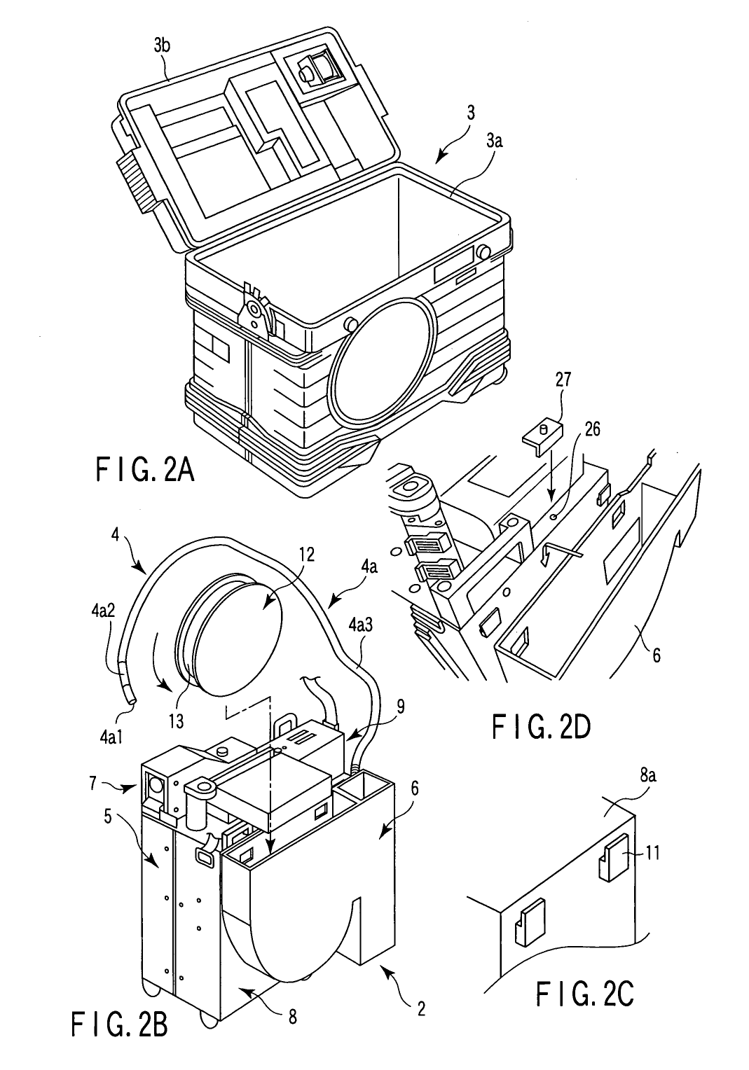

Moreover, as shown in FIG. 2A, the endoscope storage case 3 is provided with a box type case main body 3a whose upper surface is opened, and a lid 3b which opens/closes an upper surface opening of the case main body 3a. This lid 3b is rotatably connected to one side portion of the upper surface opening of the case main body 3a via a hinge portion (not shown) of the lid 3b. Furthermore, FIG. 1A shows a state in which the apparatus main body 2 is stored in the endoscope storage case 3 while opening the lid 3b.

Additionally, FIG. 2B shows the apparatus main body 2 of the endoscope apparatus 1. A scope section 4, a fixed unit 5, and a storage section 6 are mutually detachably attached to the apparatus main body 2.

Moreover, the scope section 4 is provided with an elongated insertion portion 4a having flexibility so as to be inserted into an inspection object space. The insertion portion 4a is disposed in an endmost position, and comprises a head portion 4al in which an optical observation system for observation, an optical illuminating system and the like are incorporated, a curved portion 4a2 capable of being remotely curved/operated, and an elongated flexible tube portion 4a3. Furthermore, the curved portion 4a2 is disposed between the head portion 4a1 and the flexible tube portion 4a3.

Additionally, an illuminating window for the optical illuminating system (not shown), an observation window for the optical observation system, a tip-side open end of an internal channel (treatment tool insertion path) disposed inside the insertion portion 4a and the like are disposed in the tip surface of the head portion 4a1. Furthermore, a light guide (not shown) which transmits the illuminating light to the optical illuminating system, an electric cord disposed in the optical observation system and connected, for example, to a CCD, a curved wire for bending/operating the curved portion 4a2 and the like are disposed in the insertion portion 4a.

Moreover, a power supply section 7, a light supply device 8, a recording unit 9, a base unit 4d of the scope section 4 (FIG. 1B) and the like are disposed in the fixed unit 5. A base end portion of the insertion portion 4a is connected to the base unit 4d. An electromotive angle unit, electromotive angle substrate, camera control unit and the like (not shown) are built in the base unit 4d. Furthermore, the electromotive angle unit is connected to the curved wire in the insertion portion 4a. Power units such as a driving motor for drawing/driving the curved wire are built in the electromotive angle unit. Moreover, when the electromotive angle unit is operated to draw/drive the curved wire, the curved portion 4a2 is bent/operated in a remote manner.

Furthermore, image data of an endoscopically observed image photographed by the CCD in the insertion portion 4a is transmitted to the camera control unit via the electric cord.

Additionally, as shown in FIG. 2C, a plurality of attachment members 11 for attaching the storage section are protruded from the side surface of an exterior cover 8a of the light supply device 8.

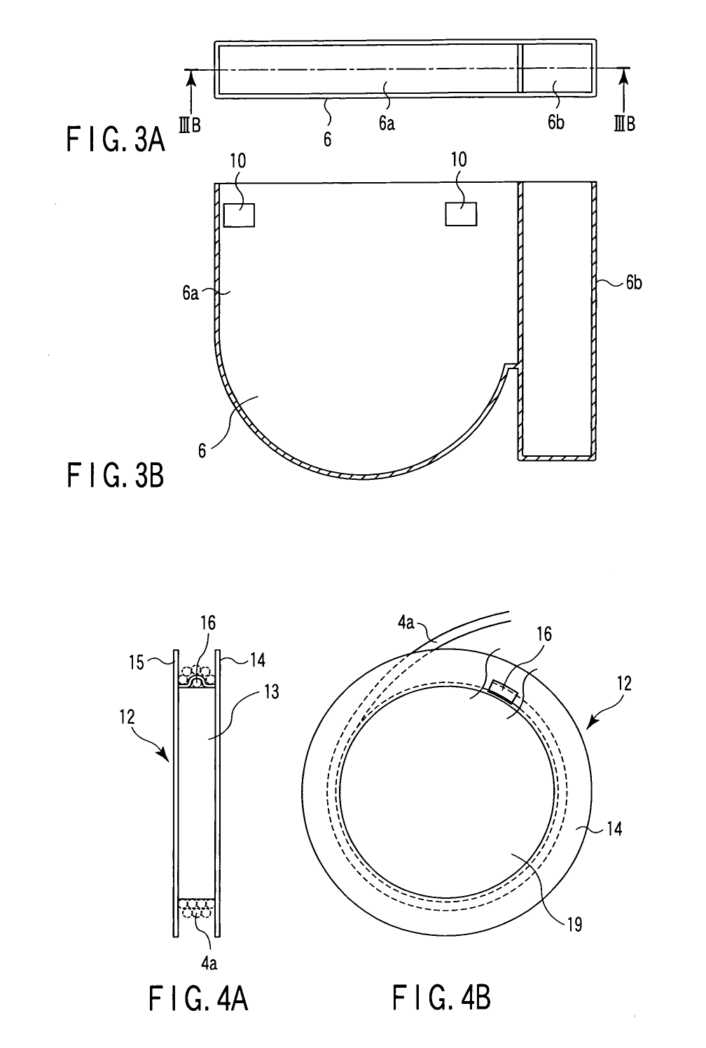

Moreover, FIGS. 3A, 3B show the storage section 6 formed of a cloth, resin, or metal material. The inside of the storage section 6 is partitioned into a plurality of chambers, two chambers in the present embodiment to form a broad scope storage box 6a (insertion portion storage section) and a narrow remote controller storage section (cables storage section) 6b. Here, a remote controller (input device) for operating the base unit 4d of the scope section 4, and a flexible cable whose one end is connected to the remote controller are stored in the remote controller storage section 6b.

A flexible tube holding member (example, a drum shape, a bobbin shape or the like) 12 is detachably stored in the scope storage box 6a. A flexible tube including the elongated insertion portion 4a is wound around the flexible tube holding member 12 manually by an operator.

FIGS. 4A, 4B show the holding member 12. It is to be noted that in the embodiment, the flexible tube includes only the insertion portion, but in embodiments described later, a tube including an intermediate connecting portion and universal cable in addition to the insertion portion and a tube including an insertion portion, monitor portion, and universal cable in addition to the insertion portion will similarly be referred to as the flexible tube.

The holding member 12 is provided with a cylindrical portion 13 formed of the cloth, resin, or metal material. Disc-shaped flanges 14, 15 are disposed on opposite ends of the cylindrical portion 13. Moreover, as shown in FIG. 4A, the cylindrical portion 13 is attached and held between the flanges 14, 15.

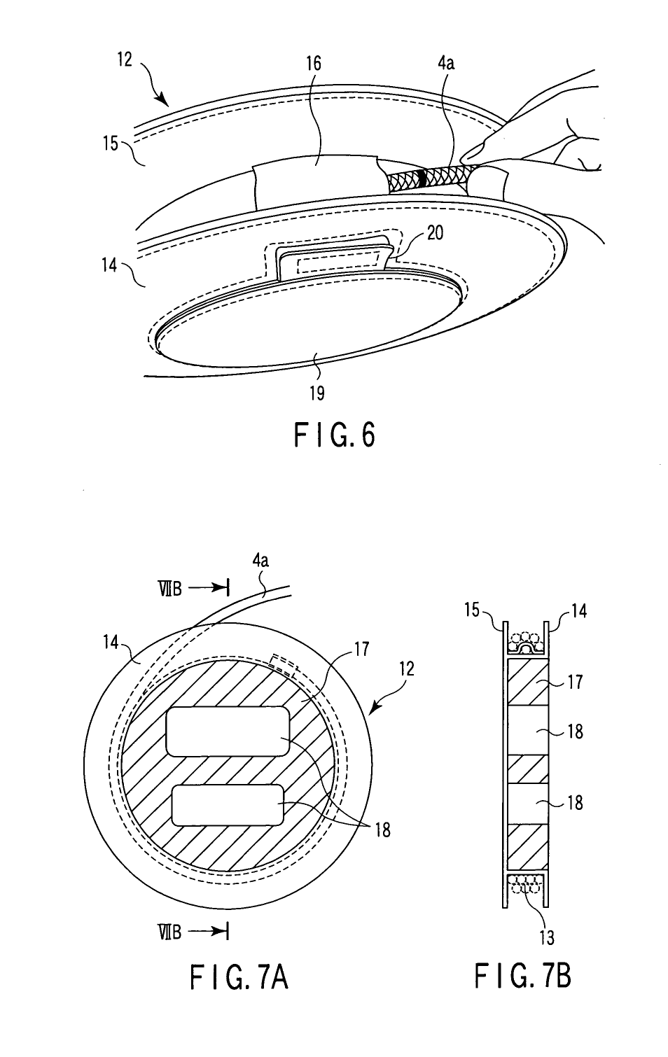

Furthermore, as shown in FIG. 6, a holder 16 of a soft member formed of cloth or rubber is attached to the outer peripheral surface of the cylindrical portion 13. The tip of the insertion portion 4a is inserted and fixed into the holder 16. Moreover, the insertion portion 4a is wound around the outer peripheral surface of the cylindrical portion 13 from the tip end of the insertion portion in a state in which the tip of the insertion portion 4a is fixed to the holder 16. Furthermore, the holding member 12 in a state in which the insertion portion 4a is wound around the cylindrical portion 13 is stored in the scope storage box 6a.

Moreover, as shown in FIGS. 7A, 7B, a cushioning material 17 formed, for example, of rubber, resin, or foaming material is disposed in a hollow portion in the cylindrical portion 13. A plurality of cutout portions 18 are formed in this cushioning material 17. Small article storage chambers in which small articles such as treatment tools and forceps are stored are formed in the respective. cutout portions 18.

Furthermore, the flange 14 on one side of the cylindrical portion 13 is opened in a portion opposite to the internal space of the cylindrical portion 13. A cover member 19 shown in FIG. 6 is openably attached to the opening. One end of this cover member 19 is fixed to an opening peripheral edge portion of the flange 14 via a hinge member (not shown). Additionally, a lingual piece 20 for handling is projected from the other end of the cover member 19.

Moreover, as shown in FIG. 3B, a plurality of engagement holes 10 are formed in the surface of the storage section 6 which is attached to/detached from the apparatus main body 2. The engagement holes 10 are arranged in positions opposite to one or a plurality of attachment members 11 fixed onto the apparatus main body 2. Moreover, the attachment members 11 of the apparatus main body 2 are caught in the engagement holes 10 of the storage section 6, and the storage section 6 is detachably attached to the attachment members 11 of the apparatus main body 2. Furthermore, as shown in FIG. 2D, a screw hole 26 is disposed in the upper surface of the apparatus main body 2 so that a storage section presser 27 can be fixed.

Moreover, as shown in FIG. 1C, a storage section 21 of a spare lamp 22 is disposed in the back surface of the lid 3b in the endoscope storage case 3 of the present embodiment. The spare lamp storage section 21 is closed by a cover 23.

Furthermore, in the present embodiment, a dead space between the endoscope storage case 3 and the apparatus main body 2 constitutes a storage space to store shoulder belt as shown in FIG. 1B.

Next, the function of the constitution will be described.

In the endoscope apparatus 1 of the present embodiment, for example, when the insertion portion 4a drawn out to the outside is stored at an end time of endoscopic inspection, the attachment members 11 are first engaged with the engagement holes 10 of the storage section 6 and fixed to the apparatus main body. Furthermore, the storage section presser 27 is fixed to the screw hole 26 of the apparatus main body 2 to press the storage section 6. Thereafter, the head portion 4a1 is inserted in the holder 16 of the cylindrical portion 13 of the holding member 12, and the operator manually winds the insertion portion 4a around the outer periphery of the cylindrical portion 13. The holding member 12 around which the insertion portion 4a is wound in an annular form in this manner is stored in the scope storage box 6a of the storage section 6.

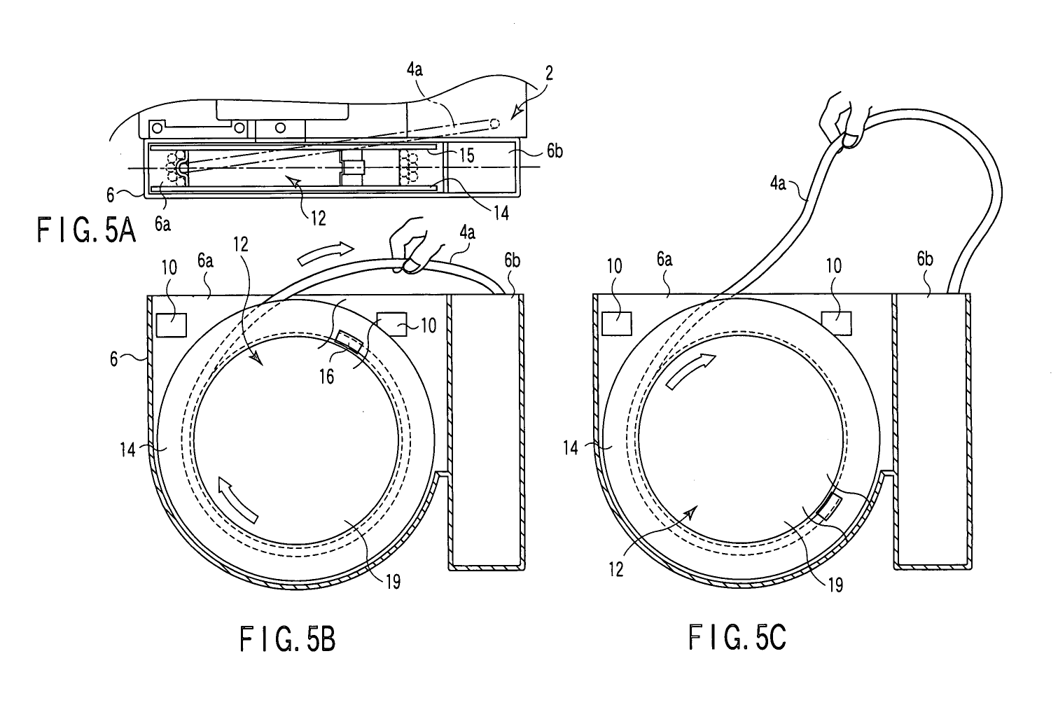

Moreover, to draw the insertion portion 4a to the outside of the holding member 12 stored in the storage section 6, as shown in FIG. 5B, the operator draws out the vicinity of the base end portion of the insertion portion 4a. Accordingly, as shown in FIG. 5C, the holding member 12 rotates in the storage section 6. Moreover, when the insertion portion 4a is further drawn to the outside, the tip of the insertion portion 4a is drawn out of the holder 16 and separated. Accordingly, the insertion portion 4a is brought into a free state.

The following effects are produced in the endoscope apparatus constituted as described above. When the holding member 12 is drawn out of the storage section 6, the holding member 12 can easily be separated from the apparatus main body 2. Therefore, in the present embodiment, it is not necessary to replace an entire assembly unit of a wind-up drum and insertion portion assembled onto the endoscope apparatus main body together as in the conventional art, and there is an effect that the insertion portion 4a can be more easily replaced.

Moreover, when the operator carries and draws out the vicinity of the base end portion of the insertion portion 4a in order to draw the insertion portion 4a to the outside, the holding member 12 rotates in the storage section 6. Accordingly, the insertion portion 4a can be easily drawn to the outside. This can be achieved by an inexpensive and simple structure.

Furthermore, as shown in FIGS. 7A, 7B, the cushioning material 17 is disposed in the hollow portion inside the cylindrical portion 13 to form the small article storage chamber in which the small articles such as the treatment tools and forceps are stored in the cutout portion 18 formed in the cushioning material 17. Therefore, since the small articles such as the treatment tools and forceps can be stored in the cutout portion 18 of the holding member 12, there is an effect that the operator conveniently uses the apparatus. Furthermore, when the tip of the insertion portion 4a is fixed to and wound around the tip holder 16, a root side of the insertion portion 4a is twisted. The generated twist can be removed, when the operator reverses the insertion portion holding member 12.

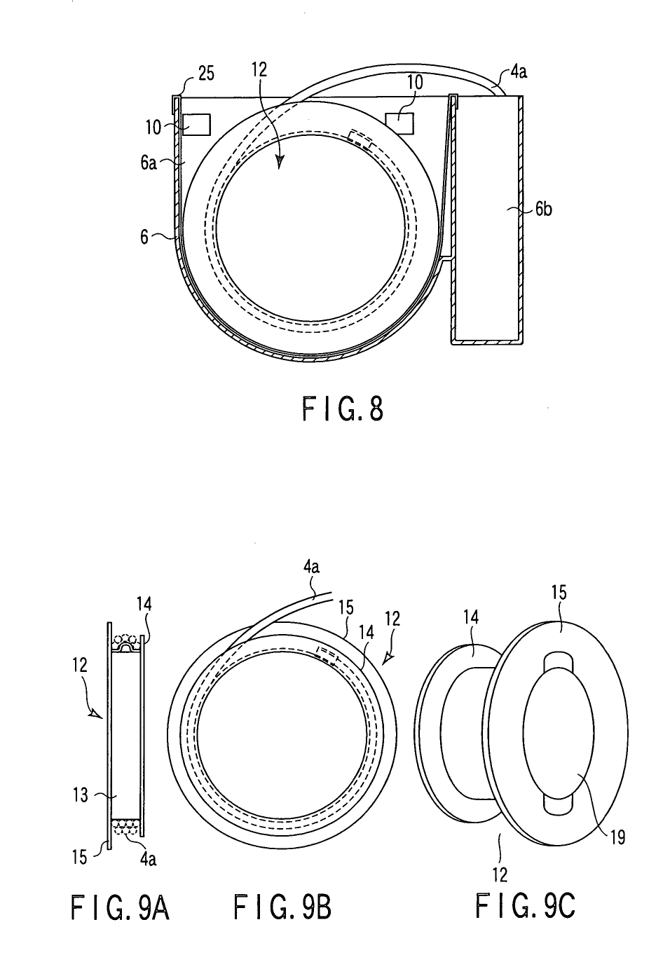

Moreover, FIG. 8 shows a second embodiment of the present invention. In the present embodiment, the constitution of the endoscope apparatus 1 of the first embodiment (refer to FIGS. 1A to 1C to 7A, 7B) is modified as follows.

That is, in the present embodiment, a sheet 25 functioning as a smooth material is disposed on a contact surface between the holding member 12 and the storage section 6. This sheet 25 is formed, for example, of resins such as polyethylene, Teflon, polyacetal, and nylon, or formed of compressed paper mixed with the resin.

There are effects that frictional forces of the holding member 12 and storage section 6 can be reduced by the sheet 25 and that wear resistances of both the components and sliding properties of the holding member 12 are enhanced in the present embodiment.

Moreover, FIGS. 9A to 9C show a third embodiment of the present invention. In the present embodiment, the constitution of the endoscope apparatus 1 of the first embodiment (see FIGS. 1A to 1C to 7A, 7B) is modified as follows. In the present embodiment, the diameter of one flange 14 of the holding member 12 is set to be smaller than that of the other flange 15.

In this case, these flanges 14, 15 have different diameters. Therefore, when the operator takes out and places the holding member 12, the holding member 12 does not fall or rolls. The insertion portion holding member 12 falls while directing upwards a flange 15 side on which the cover member 19 is disposed. Therefore, the small components stored in the cushioning material 17 can be taken out easily.

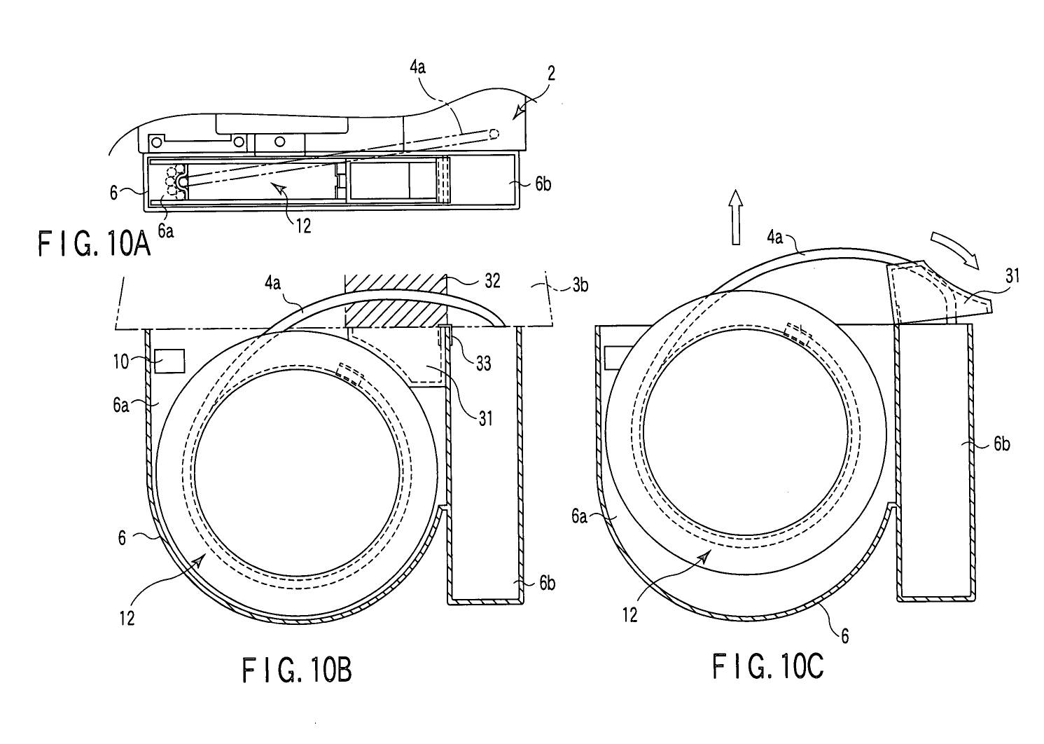

FIGS. 10A to 10C show a fourth embodiment of the present invention. In the present embodiment, the constitution of the endoscope apparatus 1 of the first embodiment (see FIGS. 1A to 1C to 7A, 7B) is modified as follows. In the present embodiment, a storage section 31 having a surface which contacts the flange outer periphery of the holding member 12 is disposed in the upper part of the storage section 6. One end of the storage section 31 is rotatably supported by a partition wall between the scope storage box 6a and the remote controller storage section 6b by the use of a hinge portion 33.

Moreover, an in-case cushioning material 32 which presses the storage section 31 from above is disposed in a position facing the storage section 31 on the back surface of the lid 3b of the endoscope storage case 3 as shown in FIG. 10B.

Next, the function of the present embodiment constituted as described above will be described. As shown in FIG. 10B, the storage section 31 contacts the in-case cushioning material 32, and the storage section 31 does not float up in a state in which the lid 3b of the endoscope storage case 3 is closed. At this time, the holding member 12 which contacts the storage section 31 does not float up. When the lid 3b of the endoscope storage case 3 is opened, and the holding member 12 is taken out of the storage section 6 as shown in FIG. 10C, the storage section 31 is rotated and taken out.

The following effect is produced in the above-described constitution. That is, even when an impact is added to the apparatus while carrying the apparatus in the present embodiment, the holding member 12 does not move in the storage section 6, and the insertion portion 4a can be prevented from being damaged.

Moreover, FIGS. 11A, 11B to 13 show a fifth embodiment of the present invention. In the present embodiment, the constitution of the endoscope apparatus 1 of the first embodiment (see FIGS. 1A to 1C to 7A, 7B) is modified as follows.

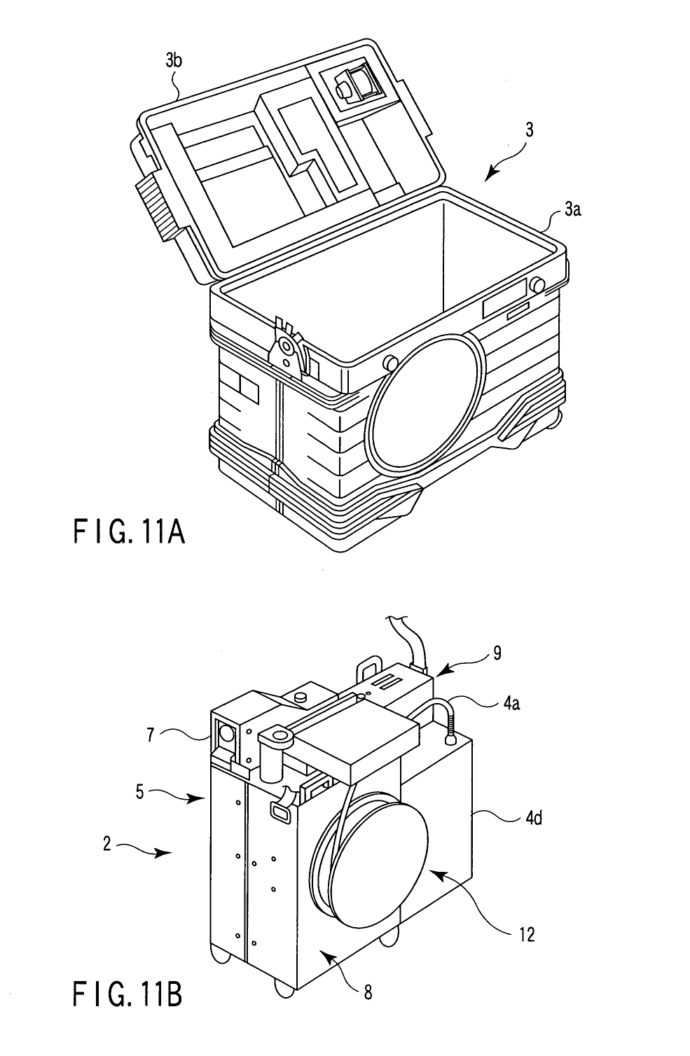

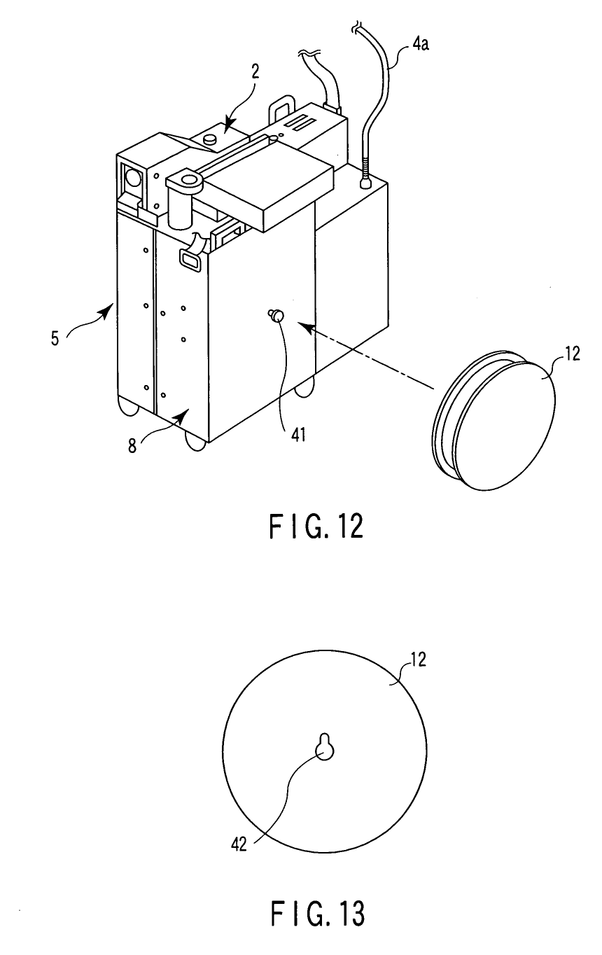

In the present embodiment, the storage section 6 of the first embodiment is omitted. As shown in FIG. 12, a support shaft 41 for attaching the holding member 12 is protruded from an end plate of the apparatus main body 2 of the endoscope apparatus 1. Furthermore, as shown in FIG. 13, an engagement hole 42 is formed in a shaft center portion of the holding member 12 and is detachably engaged with the support shaft 41.

The above-described constitution produces the following effects. That is, since the storage section 6 of the first embodiment is omitted in the present embodiment, the structure is simple and inexpensive as compared with the first embodiment. In the present embodiment, the support shaft 41 is disposed on the apparatus main body 2 side, and the engagement hole 42 is disposed on the holding member 12 side. Conversely, the engagement hole 42 may be disposed on the apparatus main body 2 side, and the support shaft 41 may be disposed on the holding member 12 side. According to this constitution, the holding member 12 can be rotated while engaged.

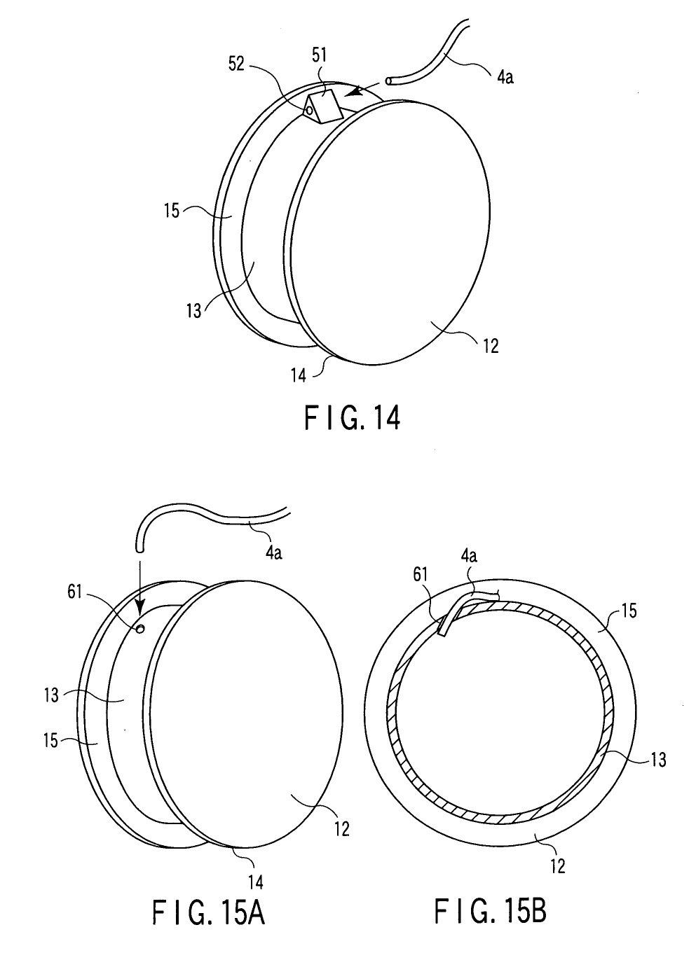

Moreover, FIG. 14 shows a sixth embodiment of the present invention. In the present embodiment, the constitution of the holding member 12 of the first embodiment (see FIGS. 1A to 1C to 7A, 7B) is modified as follows.

That is, in the present embodiment, a block-shaped tip holder 51 formed of a resin or a rubber is disposed instead of the holder 16 of the first embodiment. The tip holder 51 is provided with a hole 52 into which the insertion portion 4a can be inserted. Moreover, the tip holder 51 is disposed on the outer peripheral surface of the cylindrical portion 13 of the holding member 12.

In the present embodiment, the insertion portion 4a can be inserted in the hole 52 of the tip holder 51 to fix the tip portion of the insertion portion 4a. Therefore, the same effect as that of the first embodiment can be obtained even in the present embodiment.

Moreover, FIGS. 15A, 15B show a seventh embodiment of the present invention. In the present embodiment, the constitution of the holding member 12 of the first embodiment (see FIGS. 1A to 1C to 7A, 7B) is modified as follows.

In the present embodiment, instead of the tip holder 16 in the first embodiment, as shown in FIG. 15A, a hole 61 into which the insertion portion 4a can be detachably inserted is formed in the cylindrical portion 13 of the holding member 12. Moreover, as shown in FIG. 15B, the tip portion of the insertion portion 4a is inserted in the hole 61 of the cylindrical portion 13, so that the insertion portion 4a can be fixed. Therefore, the same effect as that of the first embodiment can be obtained even in the present embodiment.

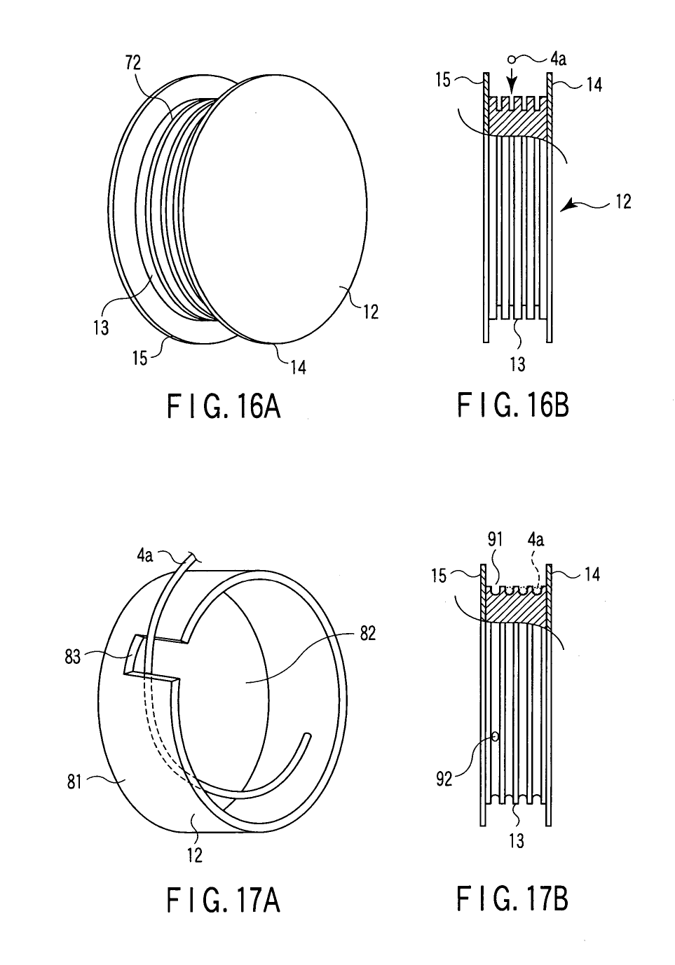

Moreover, FIGS. 16A, 16B show an eighth embodiment of the present invention. In the present embodiment, the constitution of the holding member 12 of the first embodiment (see FIGS. 1A to 1C to 7A, 7B) is modified as follows.

In the present embodiment, the cylindrical portion 13 of the holding member 12 is formed of the rubber or foaming material, and a plurality of annular grooves 72 in a peripheral direction are disposed in the outer peripheral surface of the cylindrical portion 13.

In the present embodiment, the insertion portion 4a is wound around and pushed into these grooves 72, and the whole insertion portion 4a is held by the rubber or foaming material.

This constitution produces the following effects.

In the present embodiment, the insertion portion 4a can be prevented from floating up from the cylindrical portion 13 of the holding member 12, and it is possible to prevent the insertion portions 4a from interfering with each other.

FIG. 17A shows a ninth embodiment of the present invention. In the present embodiment, the constitution of the holding member 12 of the first embodiment (see FIGS. 1A to 1C to 7A, 7B) is modified as follows.

In the present embodiment, a cutout portion 83 is disposed in a part of a cylindrical portion 81 of the holding member 12 in the first embodiment, and the whole insertion portion 4a is inserted and stored in a cylinder 82 of the cylindrical portion 81.

In the present embodiment, the insertion portion 4a is inserted via the cutout portion 83 disposed in the cylindrical portion 81, and the whole insertion portion 4a is stored in the hollow portion 82 of the cylindrical portion 81. Therefore, the insertion portion 4a can constantly be held with a defined diameter by use of a outwardly swelling force of the insertion portion 4a.

Moreover, FIG. 17B shows a tenth embodiment of the present invention. In the present embodiment, the constitution of the holding member 12 of the first embodiment (see FIGS. 1A to 1C to 7A, 7B) is modified as follows.

In the present embodiment, a groove 91 having a diameter equal to that of the insertion portion 4a is disposed in the cylindrical portion 13 of the holding member 12. A hole 92 in which the insertion portion 4a can be inserted is disposed in a part of the groove 91.

Furthermore, the insertion portion 4a is inserted into the hole 92 disposed in a part of the groove 91 from the tip portion in the present embodiment, and the insertion portion 4a is wound along the groove 91. Therefore, even in the present embodiment, the insertion portions 4a can be inhibited from interfering with each other.

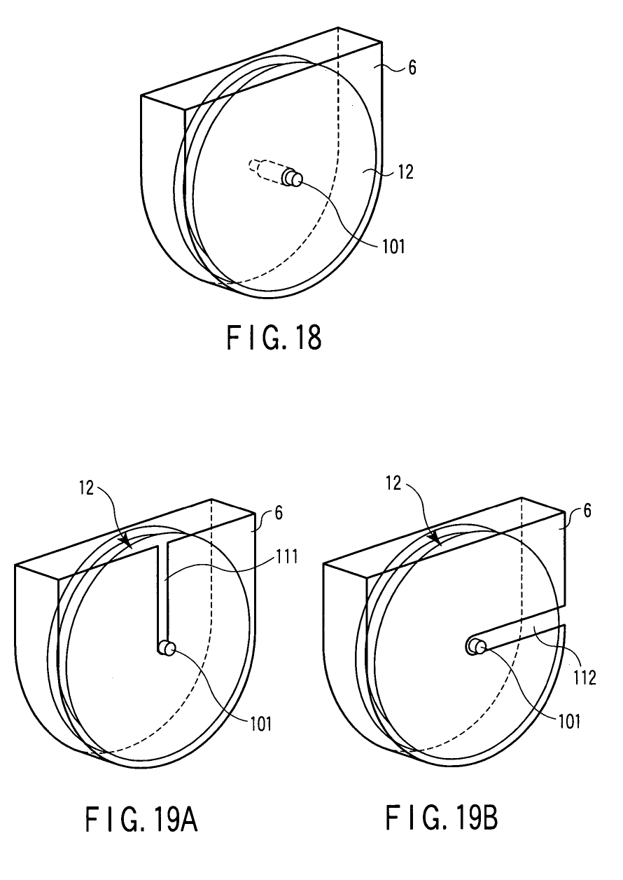

Moreover, FIG. 18 shows an eleventh embodiment of the present invention. In the present embodiment, the constitutions of the storage section 6 and holding member 12 of the first embodiment (see FIGS. 1A to 1C to 7A, 7B) are modified as follows.

In the present embodiment, the holding member 12 is held in the storage section 6 by a shaft member 101, and is rotatably attached.

Therefore, in the present embodiment, when the holding member 12 itself rotates around the shaft member 101, the winding-up and taking-out (rotation reverse to that at the winding-up time) of the insertion portion 4a are facilitated.

Moreover, FIG. 19A shows a twelfth embodiment of the present invention. In the present embodiment, the constitutions of the storage section 6 and holding member 12 of the eleventh embodiment (see FIG. 18) are modified as follows.

In the present embodiment, since a slit 111 is extended to the portion supported by the shaft member 101 from above, the holding member 12 is detachably attached to the storage section 6.

Therefore, in the present embodiment, the holding member 12 suitable for the insertion portion 4a can be selected. There is also an effect that the sheet 25 to reduce friction between the storage section 6 and the holding member 12 as described in the second embodiment (see FIG. 8) is easily replaced.

Furthermore, FIG. 19B shows a modification of the twelfth embodiment (see FIG. 19A). In the modification, a slit 112 is extended in a transverse direction in the storage section 6. It is to be noted that a slit in a slant direction may also be disposed instead of the slit 112. Additionally, to dispose the slit 112 in the transverse or slant direction, a side- wall portion needs to be opened in such a manner that the holding member 12 enters the storage section 6 on a side-wall side. Also in this case, the effect similar to that of the twelfth embodiment can be obtained. It is to be noted that a flange portion is manually rotated in order to rotate the holding member 12 in the present modification.

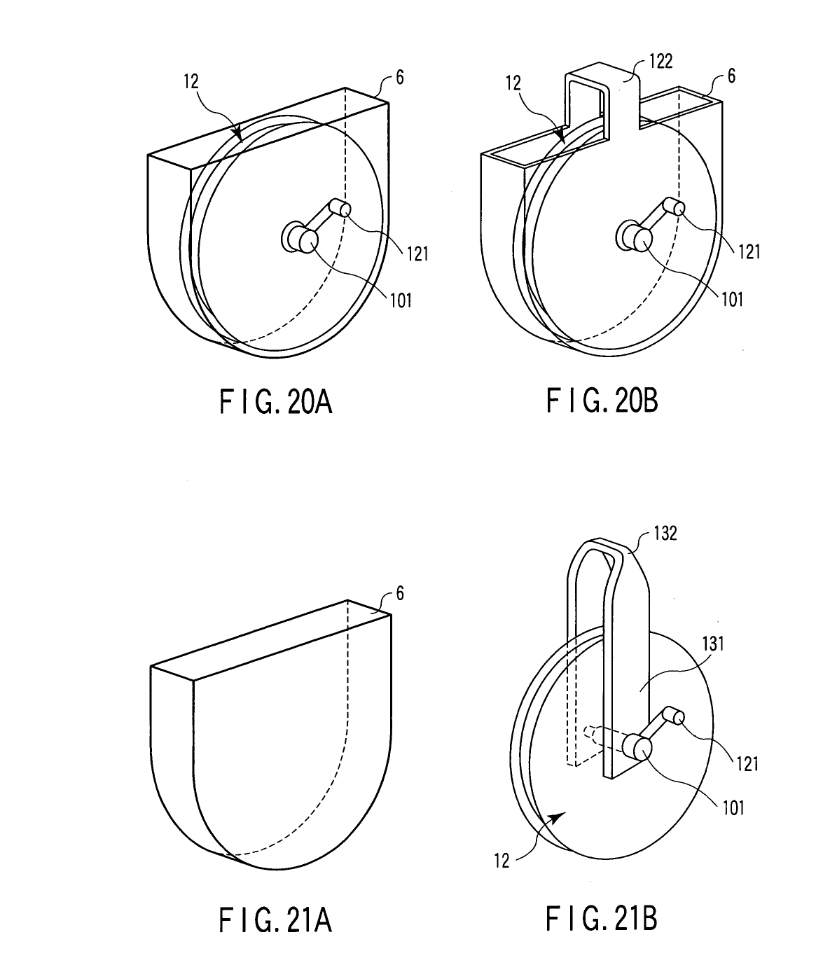

Moreover, FIG. 20 shows a thirteenth embodiment of the present invention. In the present embodiment, the shaft member 101 of the twelfth embodiment (see FIG. 19A) is connected to a handle 121, and this handle 121 achieves the rotation. It is to be noted that the slit 111 of the twelfth embodiment (see FIG. 19A) may also be combined.

In the present embodiment, there is an effect that the holding member 12 is easily rotated. It is to be noted that a grasping portion 122 may also be disposed as in a modification shown in FIG. 20B so that the storage section 6 can be taken out and used.

Moreover, FIGS. 21A, 21B show a fourteenth embodiment of the present invention. In the present embodiment, the constitutions of the storage section 6 and holding member 12 of the first embodiment (see FIGS. 1A to 1C to 7A, 7B) are modified as follows.

In the present embodiment, the holding member 12 comprises a support frame 131 which rotatably supports the member, the handle 121 which rotates the holding member 12, and a grasping portion 132 connected to the support frame 131 to grasp the whole holding member 12, and the holding member 12 is detachably attached to the storage section 6.

Therefore, in the present embodiment, the insertion portion 4a is taken out only by a portion required for the winding-up, and winding-up properties can further be enhanced.

FIG. 22A shows a fifteenth embodiment of the present invention. In the present embodiment, the constitution of the holding member 12 of the first embodiment (see FIGS. 1A to 1C to 7A, 7B) is modified as follows.

In the present embodiment, a support shaft 141 for the handle is protruded from the outer peripheral portion of the flange 14 of the holding member 12. Therefore, in the present embodiment, an operation of winding the insertion portion 4a around the outer peripheral surface of the cylindrical portion 13 of the holding member 12 can easily be performed.

FIG. 22B shows a sixteenth embodiment of the present invention. In the present embodiment, the constitution of the holding member 12 of the first embodiment (see FIGS. 1A to 1C to 7A, 7B) is modified as follows.

In the present embodiment, the support shaft 141 for the handle is protruded from the outer peripheral portion of the flange 14 of the holding member 12, and an operation lever 142 is attached to the support shaft 141. Therefore, in the present embodiment, the operation of winding the insertion portion 4a around the outer peripheral surface of the cylindrical portion 13 of the holding member 12 can easily be performed.

Moreover, FIG. 22C shows a seventeenth embodiment of the present invention. In the present embodiment, the constitution of the holding member 12 of the first embodiment (see FIGS. 1A to 1C to 7A, 7B) is modified as follows.

In the present embodiment, a support shaft 151 for the handle is protruded from a shaft center portion of the flange 14 of the holding member 12, and an operation lever 152 is attached to the support shaft 151. Therefore, in the present embodiment, the operation of winding the insertion portion 4a around the outer peripheral surface of the cylindrical portion 13 of the holding member 12 can further easily be performed.

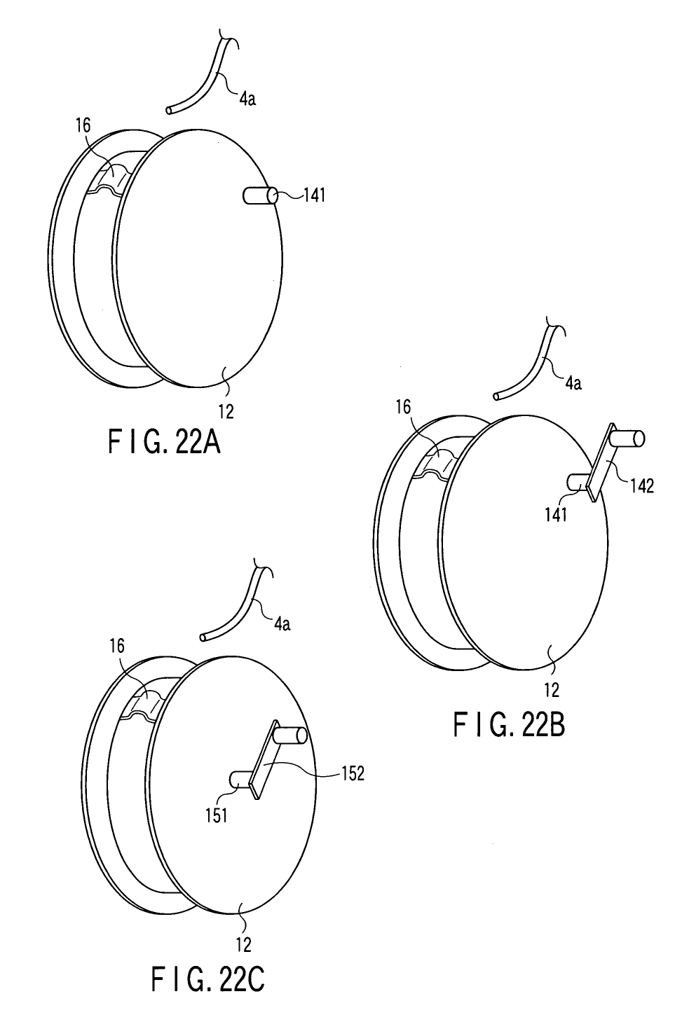

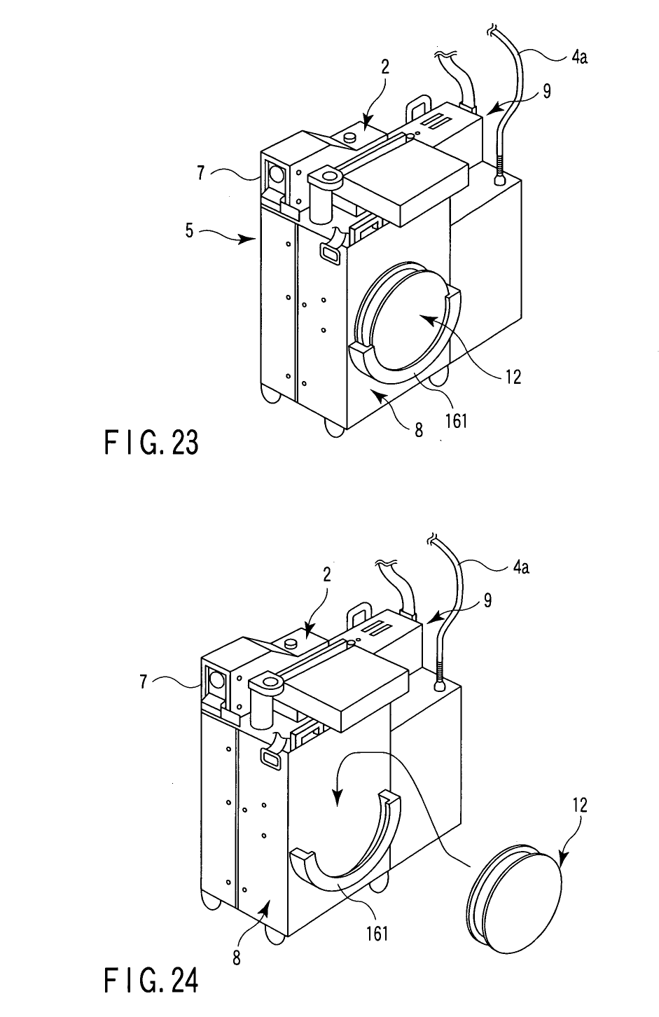

FIGS. 23 and 24 show an eighteenth embodiment of the present invention. In the present embodiment, the storage section 6 of the first embodiment (see FIGS. 1A to 1C to 7A, 7B) is modified as follows.

That is, in the present embodiment, a substantially circular receiving member 161 is disposed on the outer wall surface of the apparatus main body 2, and the holding member 12 is supported by the receiving member 161. The effect similar to that of the first embodiment can be obtained even in the present embodiment.

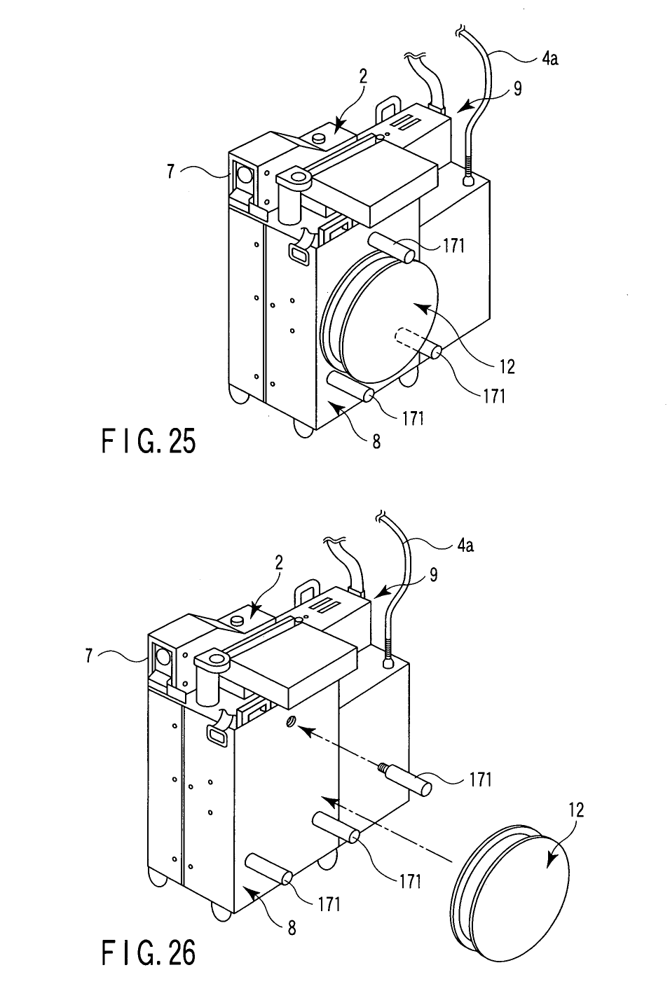

FIGS. 25 and 26 show a nineteenth embodiment of the present invention. In the present embodiment, the storage section 6 of the first embodiment (see FIGS. 1A to 1C to 7A, 7B) is modified as follows.

In the present embodiment, a plurality of pins 171 are protruded from the outer wall surface of the apparatus main body 2, and the holding member 12 is held between the pins 171. The effect similar to that of the first embodiment can be obtained even in the present embodiment.

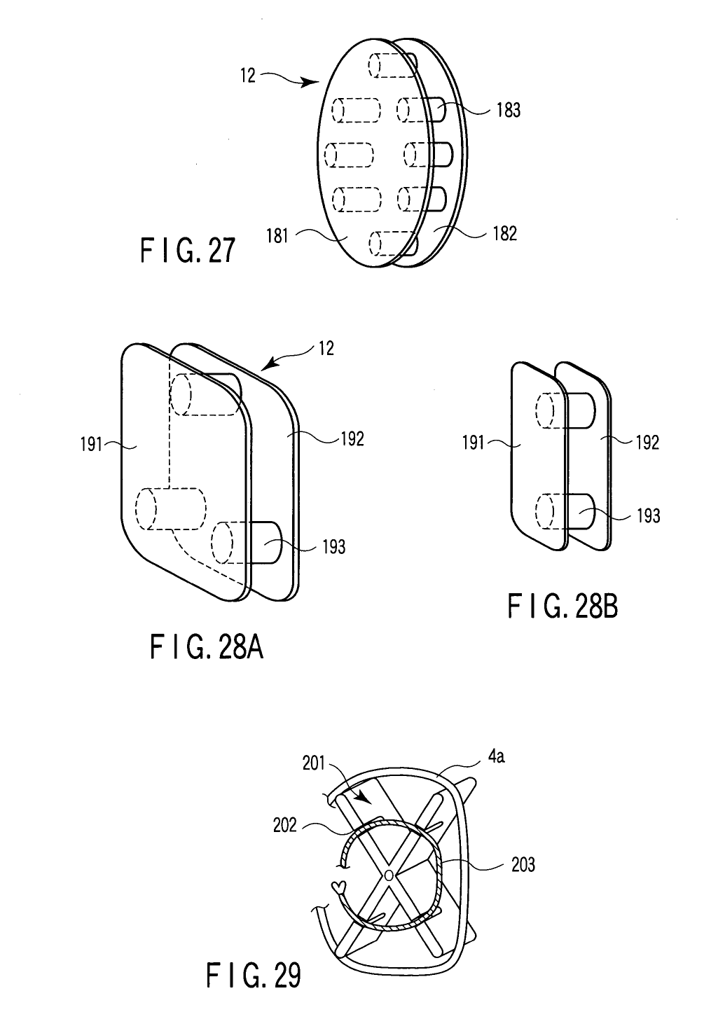

Moreover, FIG. 27 shows a twentieth embodiment of the present invention. In the present embodiment, the holding member 12 of the first embodiment (see FIGS. 1A to 1C to 7A, 7B) is modified as follows.

In the present embodiment, a plurality of support bars 183 are arranged along a peripheral direction between two circular plates 181, 182. Moreover, these support bars 183 form a winding-up portion around which the insertion portion 4a is wound to hold the insertion portion. It is to be noted that the plurality of support bars 183 are not necessarily disposed in a cylindrical shape, and may also be disposed in prism shapes such as a triangular prism and a quadratic prism.

Moreover, FIG. 28A shows a twenty-first embodiment of the present invention. In the present embodiment, the holding member 12 of the first embodiment (see FIGS. 1A to 1C to 7A, 7B) is modified as follows.

In the present embodiment, a plurality of (three herein) support bars 193 are arranged along the peripheral direction between two side plates 191, 192. Moreover, these support bars 193 form the winding-up portion around which the insertion portion 4a is wound to hold the insertion portion. It is to be noted that three or more support bars 193 may be disposed. The side plates 191, 192 may have rectangular, elliptic, or curved shapes. Two support bars 193 may also be disposed as in the modification shown in FIG. 28B. In this case, the outer diameter of the support bars 193 is preferably large.

Moreover, FIG. 29 shows a twenty-second embodiment of the present invention. In the present embodiment, the holding member 12 of the first embodiment (see FIGS. 1A to 1C to 7A, 7B) is modified as follows.

In the present embodiment, an X-shaped holding member 201 is disposed. Cutouts 202 are formed in side portions of the drum 201. Moreover, the insertion portion 4a is wound around the outer peripheral portion of the wind-up drum 201, and a treatment tool 203 is engaged with the cutouts 202.

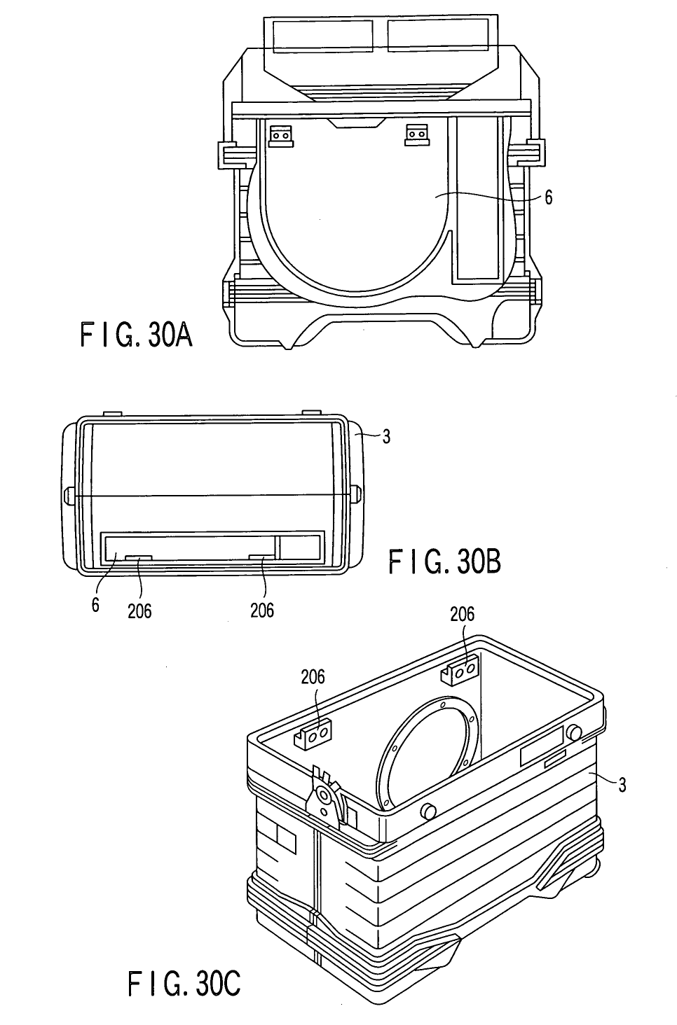

Moreover, FIGS. 30A, 30B, 30C show a twenty-third embodiment of the present invention. In the present embodiment, the attachment members 11 of the first embodiment (see FIGS. 1A to 1C to 7A, 7B) is modified as follows. That is, attachment members 206 to be engaged with the respective engagement holes 10 disposed in the storage section 6 are attached to the case 3.

The above-described constitution produces the following effect. Only the apparatus main body 2 is taken out of the endoscope case 3 and used at a movement or inspection time at which the storage section 6 is not required. Accordingly, the apparatus can further be miniaturized and lightened.

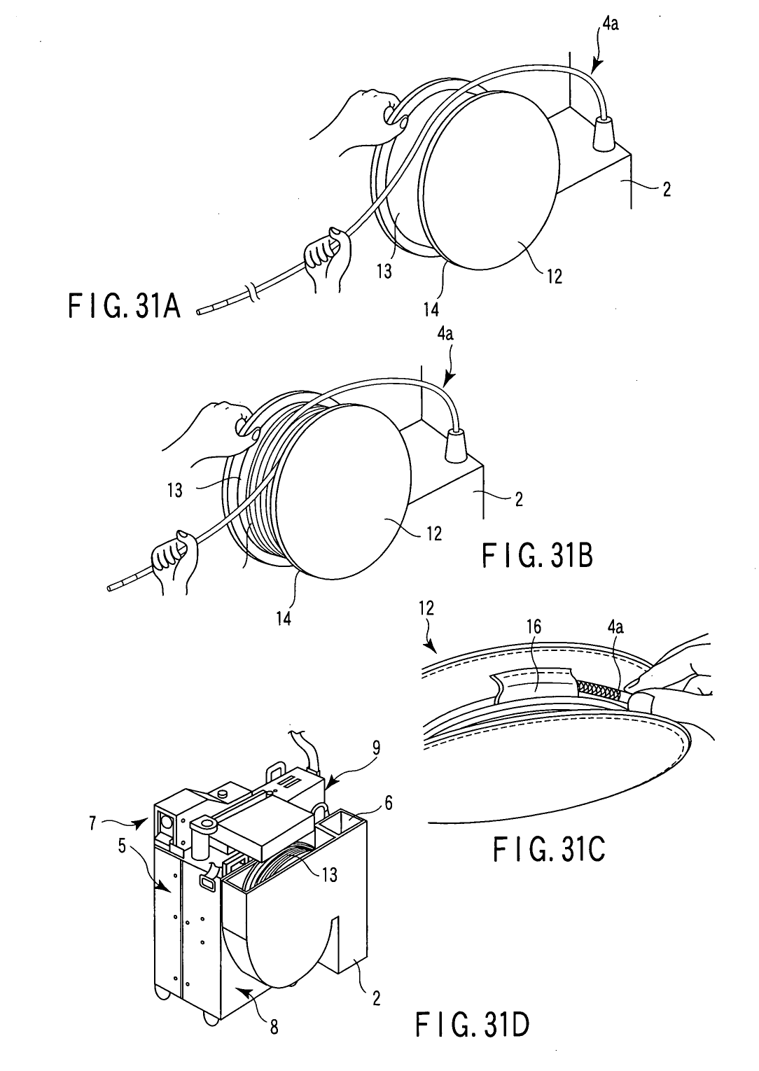

Next, a twenty-fourth embodiment of the present invention will be described. FIGS. 31A to 31D are diagrams showing the constitution example of the endoscope apparatus of the twenty-fourth embodiment. It is to be noted that for constituting portions of the present embodiment, the same portions as those shown in FIGS. 2A to 2C are denoted with the same reference numerals and the description thereof is omitted.

In the endoscope apparatus 1 of the first embodiment, as shown in FIG. 2B, after the tip of the insertion portion 4a is inserted and fixed into the holder 16 disposed on the holding member 12, the insertion portion 4a (scope section 4) is manually wound/attached. On the other hand, in the present embodiment, as shown in FIG. 31A, the insertion portion 4a starts to be wound/attached from a root side, that is, from a side connected to the light supply device 8. Moreover, when the insertion portion 4a is wound around to the tip as shown in FIG. 31B, the tip portion of the insertion portion 4a is inserted and fixed into the holder 16 disposed over the side surface of the flange 14 (15) of the holding member 12 as shown in FIG. 31C. The holding member 12 around which the insertion portion 4a is wound/attached is stored in the storage section 6 as shown in FIG. 31D.

According to the present embodiment, since the insertion portion 4a starts to be wound/attached around the holding member 12 from the root, an operation of hauling in the tip of the insertion portion 4a is eliminated. The insertion portion 4a is wound/attached around the holding member in a state in which the tip portion of the insertion portion 4a is released, and therefore twists can be prevented from being generated during the winding/attaching.

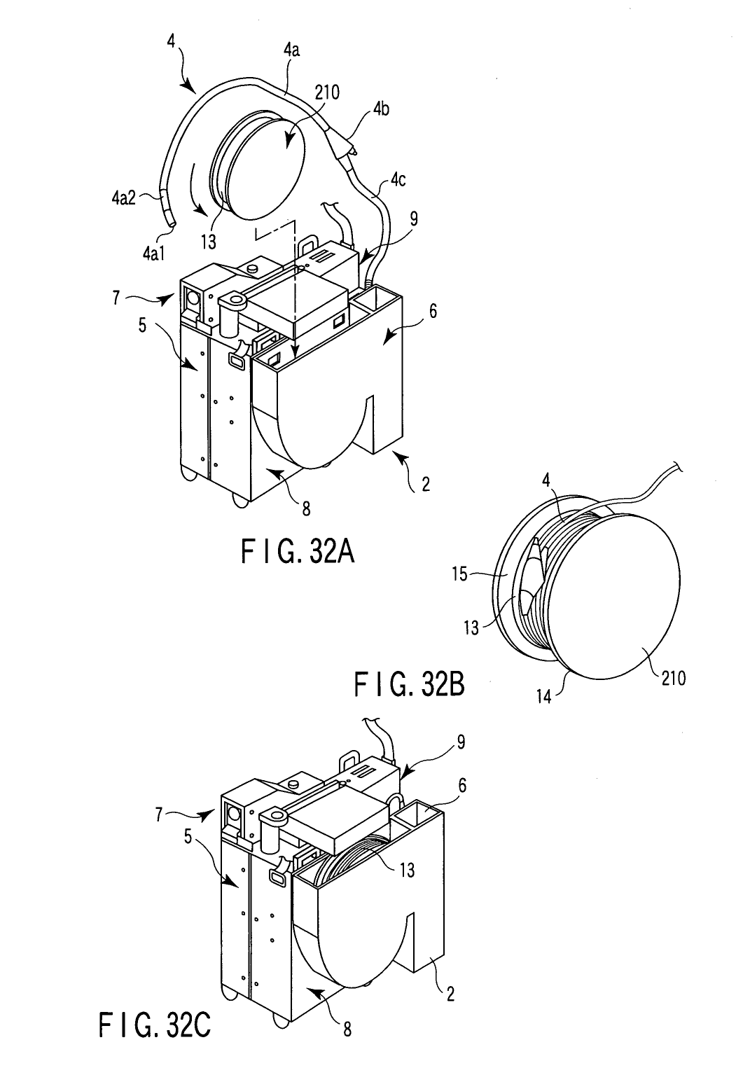

Next, a twenty-fifth embodiment of the present invention will be described. FIGS. 32A to 32C are diagrams showing the constitution example of the endoscope apparatus of the twenty-fifth embodiment.

The scope section 4 including the insertion portion 4a is disposed in the endoscope apparatus 1 of the first embodiment shown in FIGS. 2A to 2C. On the other hand, in the present embodiment, the flexible tube comprising the insertion portion 4a, a universal cable 4c, and an intermediate connecting portion 4b connecting the insertion portion to the universal cable, that is, the scope section 4 is disposed in the endoscope apparatus. In the constituting portions of the present embodiment, the same portions as those shown in FIGS. 2 are denoted with the same reference numerals and the description thereof is omitted.

In the present embodiment, as shown in FIG. 32B, the scope section 4 including the insertion portion 4a, intermediate connecting portion 4b, and universal cable 4c is wound/attached around a holding member 210. During the winding-up, the scope section 4 may also be started to be wound/attached from either the tip side or the root side. Moreover, the holding member 210 around which the scope section 4 is wound/attached is stored in the storage section 6 as shown in FIG. 32C.

According to the present embodiment, not only the scope section 4 including only the insertion portion 4a but also the scope section 4 including the insertion portion 4a and the intermediate connecting portion 4b and the universal cable 4c can similarly be wound/attached around the holding member 210 and stored in the storage section, and the effect similar to that of the first embodiment can be obtained.

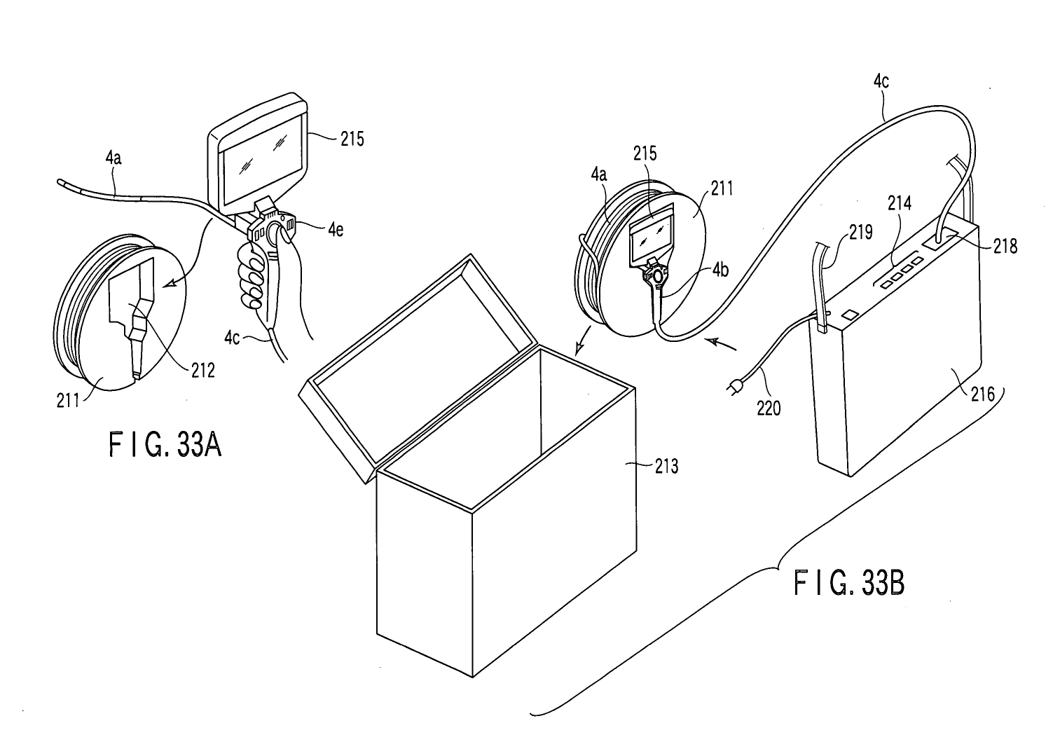

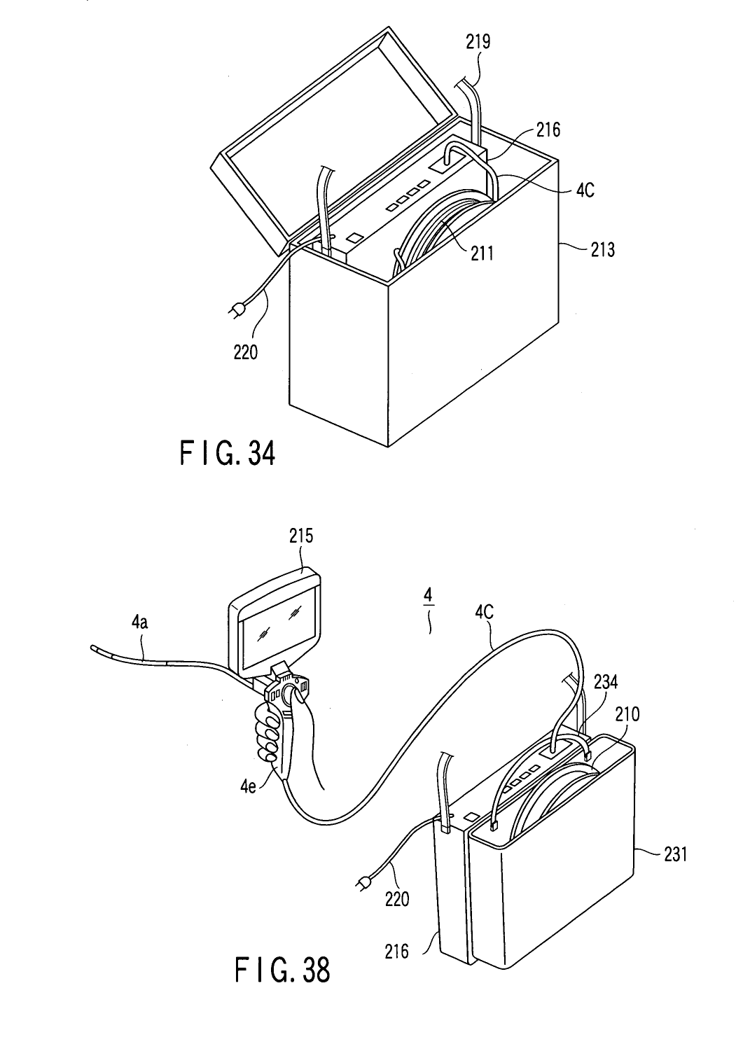

Next, a twenty-sixth embodiment of the present invention will be described. FIGS. 33A, 33B, and 34 are diagrams showing the constitution example of the endoscope apparatus of the twenty-sixth embodiment.

The endoscope apparatus comprises an apparatus main body 216 into which the constituting elements of the endoscope are integrally assembled, and a holding member 211 around which the scope section 4 detachably attached to the apparatus main body 216 is wound. The apparatus main body 216 and holding member 211 are stored in an endoscope storage case 213 provided with an opening/closing lid at a movement or storage time.

As shown in FIG. 33A, the scope section 4 comprises the insertion portion 4a, the universal cable 4c, and an operation portion 4e connecting the insertion portion to universal cable. The operation portion 4e is provided with a monitor section 215 in which an observed image, operation information and the like are displayed. As shown in FIG. 33B, an operation panel 214, a connector section 218 electrically and optically connected to the universal cable 4c of the scope section 4, a shoulder belt 219 for shouldering the apparatus at the movement time, and a power supply cable 220 are disposed on the upper surface of the apparatus main body 216. For the holding member 211, as shown in FIG. 33A, a fitting portion 212 is disposed to extend through a flange side surface in which the operation portion 4e and monitor section 215 are fitted.

In this constitution, when the flexible tube, that is, the scope section 4 is wound around the holding member 211, first the insertion portion 4a is passed through the fitting portion 212, and the monitor section 215 is fitted/fixed in the fitting portion 212. The insertion portion 4a is wound/attached around the holding member 211 from a monitor section 215 side, and the tip of the insertion portion provided with the holder 16 of the twenty-fourth embodiment is inserted/fixed. The universal cable 4c is also appropriately wound/attached around the holding member 211 from the monitor section 215 side, and is stored into the endoscope storage case 213 together with the apparatus main body 216 as shown in FIG. 34.

According to the present embodiment, when the scope section 4 provided with the monitor section 215 is stored in the endoscope storage case 213, the monitor section 215 and operation portion 4e are fitted/fixed in the holding member 211, and therefore the inserting/detaching of the holding member 211 with respect to the endoscope storage case 213 is facilitated.

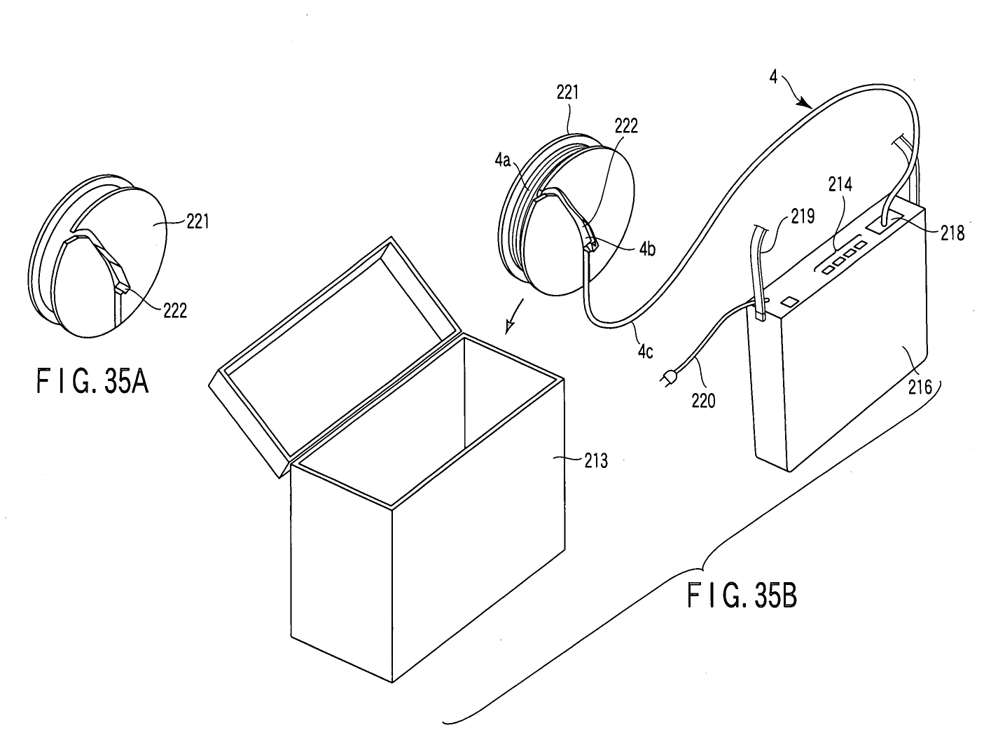

Next, a twenty-seventh embodiment of the present invention will be described. FIGS. 35A, 35B are diagrams showing the constitution example of the endoscope apparatus of the twenty-seventh embodiment.

In the present embodiment, the apparatus main body 216 into which the constituting elements of the endoscope are integrally assembled, and the flexible tube comprising the insertion portion 4a and the universal cable 4c and the intermediate connecting portion 4b connecting the insertion portion to the universal cable, that is, the scope section 4 are disposed in the endoscope apparatus. For the constituting portions of the present embodiment, the same portions as those shown in FIGS. 33A, 33B are denoted with the same reference numerals and the description thereof is omitted.

In the present embodiment, as shown in FIG. 35B, the scope section 4 including the insertion portion 4a, intermediate connecting portion 4b, and universal cable 4c is wound/attached around a holding member 221. For the holding member 221, as shown in FIG. 35A, a fitting portion 222 having a concave shape is disposed in the flange side surface in order to fit the intermediate connecting portion 4b in the fitting portion. The apparatus main body 216 and holding member 221 are stored in the endoscope storage case 213 provided with the opening/closing lid at the movement or storage time.

In the constitution, when the flexible tube, that is, the scope section 4 is wound/attached around the holding member 221, first the intermediate connecting portion 4b is fitted/fixed into the fitting portion 222. The insertion portion 4a is wound/attached around the holding member 221 from the intermediate connecting portion 4b side, and the tip of the insertion portion provided with the holder 16 of the twenty-fourth embodiment is inserted/fixed. The universal cable 4c is also appropriately wound/attached around the holding member 221 from the intermediate connecting portion 4b side, and stored in the endoscope storage case 213 together with the apparatus main body 216.

According to the present embodiment, when the scope section 4 provided with the intermediate connecting portion 4b is stored in the endoscope storage case 213, the intermediate connecting portion 4b is fitted/fixed in the holding member 221, and therefore the inserting/detaching of the holding member 221 with respect to the endoscope storage case 213 is facilitated.

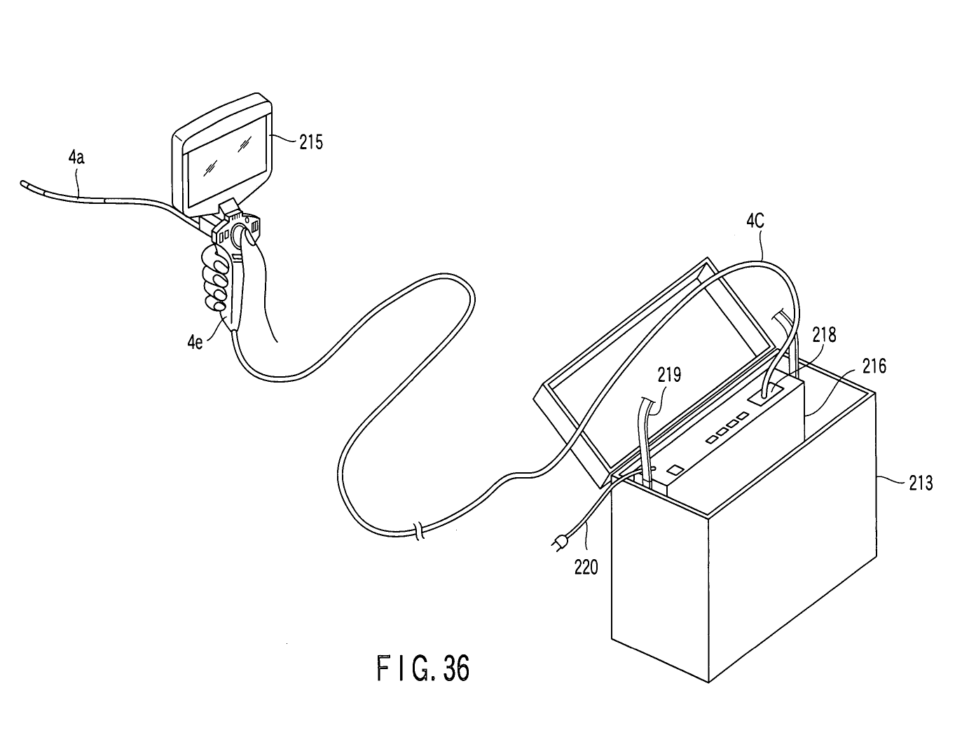

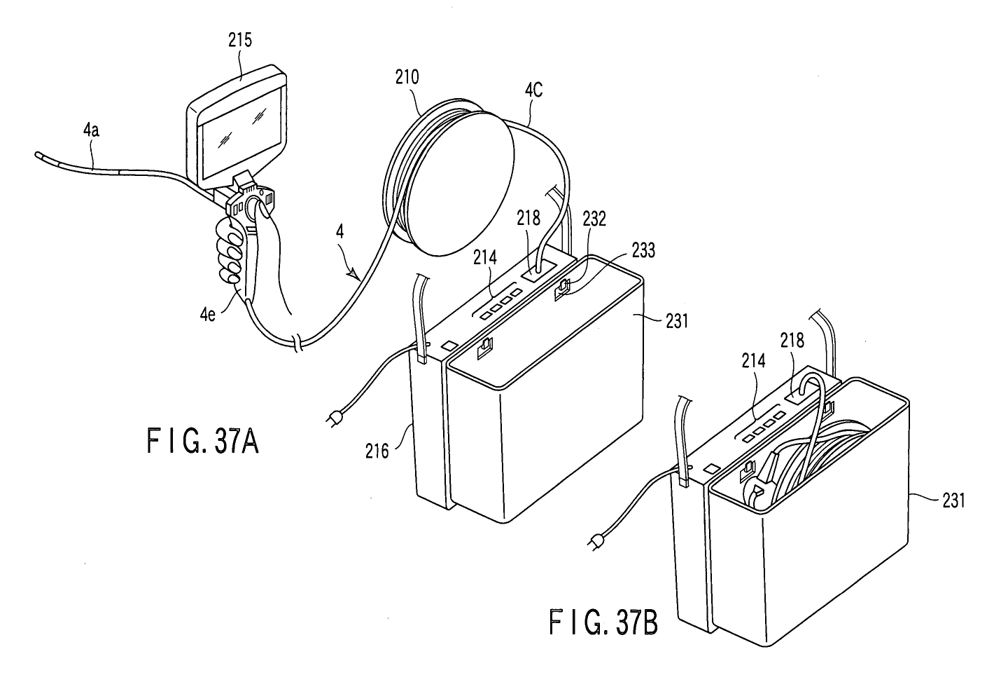

Next, a twenty-eighth embodiment of the present invention will be described. FIGS. 36, 37A, 37B are diagrams showing the constitution example of the endoscope apparatus of the twenty-eighth embodiment.

When observation by the endoscope apparatus is performed, an inspection place is small, restrictions are imposed, and the apparatus main body 216 cannot be carried in the vicinity of an inspection portion in some case. In this case, as shown in FIG. 36, a very long universal cable 4c is a convenient specification. However, when the universal cable 4c lengthens, the operation has difficulty in drawing out or drawing around/storing the cable, and it becomes difficult to handle the cable.

To solve the problem, in the present embodiment, as shown in FIG. 37A, the universal cable 4c is wound around the holding member 210 and used. Here, the scope section 4 comprises the insertion portion 4a, universal cable 4c, operation portion 4e, and monitor section 215.

The holding member 210 is stored in a storage section 231 at the movement or storage time.

In the present embodiment, as shown in FIG. 37A, the universal cable 4c is wound/attached around the holding member 210. Here, the scope section 4 comprises the insertion portion 4a, universal cable 4c, operation portion 4e, and monitor section 215.

The holding member 210 is stored in the storage section 231 at the movement or storage time. The holding member 210 may be provided with the fitting portion in which the operation portion including the intermediate connecting portion and monitor section such as the holding member 211 or 221 described with reference to FIG. 33 or 35 is fitted and fixed.

Moreover, as shown in FIG. 37A, a plurality of hook portions 232 are disposed on the side surface of the apparatus main body 216. Windows 233 for attachment are opened in the side surface of the storage section 231 so that the hook portions 232 are fitted in the windows. When the holding member 210 is attached to the hook portions 232, the apparatus can be integrally carried.

To store the scope section 4 in the storage section 231, the holding member 210 around which the universal cable 4c is wound is stored in the storage section 231, the operation portion 4e and monitor section 215 are disposed in the upper part of the storage section, the insertion portion is inserted in a gap, and the scope section can thus be stored. After further winding/attaching the insertion portion 4a onto the universal cable 4c wound around the holding member 210, the holding member 210 may also be stored in the storage section 231 in such a manner that the operation portion 4e and monitor section 215 are positioned above.

Next, a twenty-ninth embodiment of the present invention will be described. FIG. 38 is a diagram showing the constitution example of the endoscope apparatus of the twenty-ninth embodiment.

The apparatus main body 216 and storage section 231 are preferably integrally engaged during the movement. Then, as shown in FIG. 38, a string 234 is disposed on the upper part of the storage section 231, the scope section 4 is passed under the string, and the string is hooked on and engaged with the root portion of the universal cable 4c. A length of the string 234 is adjusted beforehand in such a manner that the side surface of the apparatus main body 216 abuts on that of the storage section 231. A length adjustment portion may also be disposed. It is to be noted that in the example, the string 234 is caught on the root side of the scope section 4. However, when a load is applied onto the scope section 4, a hook for exclusively hooking the string 234 may be separately disposed on the apparatus upper surface.

Therefore, according to the present embodiment, the apparatus main body 216 and storage section 231 can be easily integrated, and therefore conveniently carried.

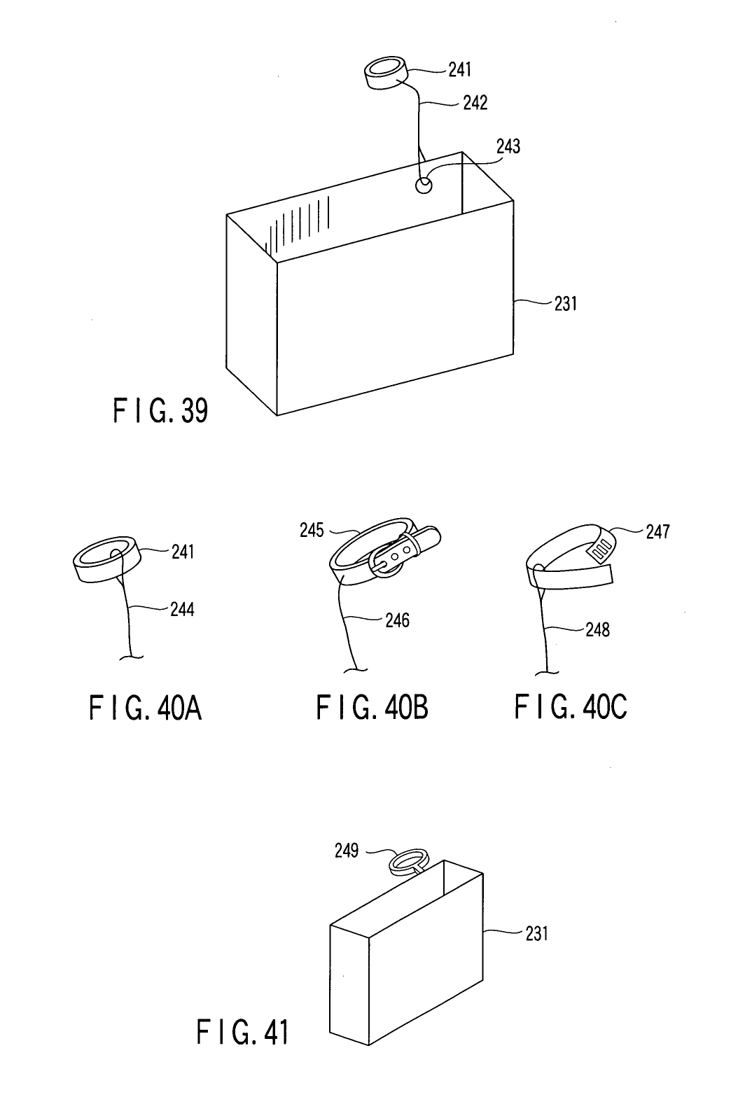

Next, FIGS. 39, 40A to 40C, 41 show modifications in the engagement of the apparatus main body 216 with the storage section 231.

In the first modification shown in FIG. 39, an annular portion 241 inside which the universal cable 4c is passed is connected to the storage section 231 via a string 242. This string 242 is connected to the annular portion 241 by the use of a solder, adhesive or the like, and is connected to the storage section 231 via an opened hole 243.

In the second modification shown in FIG. 40A, a string 244 is tied onto the annular portion 241, for example, by a free knot forming method. In the third modification shown in FIG. 40B, the scope section 4 comprises the intermediate connecting portion 4b or the operation portion 4e and the monitor section 215, and a band portion 245 whose diameter is adjustable is used instead of the annular portion 241. In the fourth modification shown in FIG. 40C, similarly instead of the annular portion 241, a annular portion 247 may also be constituted by the use of Magic Tape (registered trademark) or the like.

Furthermore, as the fifth modification shown in FIG. 41, an annular portion 249 may directly be disposed in the storage section 231.

According to these modifications, the apparatus main body 216 and storage section 231 are integrated, and can easily be carried in the same manner as in the twenty-ninth embodiment.

Furthermore, the present invention is not limited to the above-described embodiments, and can, needless to say, be variously modified without departing from the scope of the present invention. For example, the scope section which is the flexible tube may comprise only the insertion portion or may also comprise the intermediate connecting portion and universal cable in addition to the insertion portion or may comprise the operation portion, monitor section, and universal cable in addition to the insertion portion. These scope sections can easily be applied to any of the first to twenty-ninth embodiments and the first to fifth modifications.

According to the present invention, the holding member around which the flexible tube including the insertion portion or the insertion portion and universal cable is wound is detachably attached to the endoscope storage case and can easily be replaced. There can be provided the endoscope apparatus which is easily portable and which is capable of inexpensively constituting the whole system.

What is claimed is:

1. An endoscope apparatus comprising:
an endoscope including an elongated flexible tube having flexibility such that a part of the flexible tube is inserted in at least an inspection object space;
an endoscope apparatus main body connected to the endoscope for use; and
a flexible tube holding member which is detachably attached to the endoscope apparatus main body and around which the flexible tube is wound to hold the flexible tube.

2. An endoscope apparatus comprising:
an endoscope apparatus main body connected to an endoscope including an elongated flexible tube having flexibility such that a part of the flexible tube is inserted in at least an inspection object space;
a flexible tube holding member around which the flexible tube is wound to hold the flexible tube; and
a storage section which stores the flexible tube holding member and which is detachably attached to the endoscope apparatus main body.

3. The endoscope apparatus according to claim 1, wherein the flexible tube comprises a head portion in which an optical system in an endmost position is incorporated, a curved portion capable of being bent/operated in a remote manner, and an elongated flexible tube portion.

4. The endoscope apparatus according to claim 2, wherein the flexible tube comprises a head portion in which an optical system in an endmost position is incorporated, a curved portion capable of being bent/operated in a remote manner, and an elongated flexible tube portion.

5. The endoscope apparatus according to claim 3, wherein the flexible tube comprises the insertion portion, further a universal cable which has flexibility and which transmits an illuminating light and an electric signal, and an intermediate connecting portion which connects the insertion portion to the universal cable.

6. The endoscope apparatus according to claim 4, wherein the flexible tube comprises the insertion portion, further a universal cable which has flexibility and which transmits an illuminating light and an electric signal, and an intermediate connecting portion which connects the insertion portion to the universal cable.

7. The endoscope apparatus according to claim 3, wherein the flexible tube comprises the insertion portion, further a universal cable which has flexibility and which transmits an illuminating light and an electric signal, an operation portion which connects the insertion portion to the universal cable and which controls an operation of the insertion portion, and a monitor portion which displays an image photographed by the head portion by operation of the operation portion and image information.

8. The endoscope apparatus according to claim 4, wherein the flexible tube comprises the insertion portion, further a universal cable which has flexibility and which transmits an illuminating light and an electric signal, an operation portion which connects the insertion portion to the universal cable and which controls an operation of the insertion portion, and a monitor portion which displays an image photographed by the head portion by operation of the operation portion and image information.

9. The endoscope apparatus according to claim 2, wherein the storage section is a container which contains the flexible tube holding member.

10. The endoscope apparatus according to claim 2, wherein the storage section is a support shaft which detachably supports the flexible tube holding member with respect to the endoscope apparatus main body.

11. The endoscope apparatus according to claim 1, wherein the flexible tube holding member has a rotatable drum or bobbin shape which rotates the flexible tube with a defined diameter.

12. The endoscope apparatus according to claim 2, wherein the flexible tube holding member has a rotatable drum or bobbin shape which rotates the flexible tube with a defined diameter.

13. The endoscope apparatus according to claim 1, wherein the flexible tube holding member comprises a rotary member around which the flexible tube is wound from a tip side.

14. The endoscope apparatus according to claim 2, wherein the flexible tube holding member comprises a rotary member around which the flexible tube is wound from a tip side.

15. The endoscope apparatus according to claim 1, wherein the flexible tube holding member comprises a rotary member around which the flexible tube is wound from a root side connected to the apparatus main body.

16. The endoscope apparatus according to claim 2, wherein the flexible tube holding member comprises a rotary member around which the flexible tube is wound from a root side connected to the apparatus main body.

17. The endoscope apparatus according to claim 5, wherein the flexible tube holding member comprises a first fitting portion which is capable of engaging with and storing the intermediate connecting portion connecting the insertion portion to the universal cable and which has a concave shape.

18. The endoscope apparatus according to claim 6, wherein the flexible tube holding member comprises a first fitting portion which is capable of engaging with and storing the intermediate connecting portion connecting the insertion portion to the universal cable and which has a concave shape.

19. The endoscope apparatus according to claim 7, wherein the flexible tube holding member comprises a second fitting portion which is capable of engaging with and storing the operation portion connecting the insertion portion to the universal cable and the intermediate connecting portion including the monitor portion.

20. The endoscope apparatus according to claim 8, wherein the flexible tube holding member comprises a second fitting portion which is capable of engaging with and storing the operation portion connecting the insertion portion to the universal cable and the intermediate connecting portion including the monitor portion.

21. The endoscope apparatus according to claim 2, wherein the storage section comprises an engagement portion which engages with the flexible tube and engages with the endoscope apparatus main body and is integrally movable.
